SEQUENCE LISTING (63 sequences)

SEQ ID NO: 1 - DNA, 145 bp, synthetic construct
  source 1..145: mol_type = other DNA
gttctgggcgcagggaggcggcggggggctgctgctgaccgcctcgcagcgctggccgggctccgggaggagggccccggcgggtggcggcgcaggagcccgaggggacagaccgggcggtggcgggggcggcgggggtggtggc

SEQ ID NO: 2 - DNA, 182 bp, synthetic construct
  source 1..182: mol_type = other DNA
gcgggggcccagggtcccggcaggccgcgtagcgctgcacggtgcacgccgcgcgccgcccgaagcccgcctccggctggaagctgctctctcgcctctggccgccggcgtagtacccgggcgagtggtcgctgggtaggtaatcgctctgtgaatattcctcgcatggagggaacttgggg

SEQ ID NO: 3 - DNA, 174 bp, synthetic construct
  source 1..174: mol_type = other DNA
gctcgcggctgcagtacccagacacctggtgcttcatcgactggaccaccaacgtgacggcgcacgccgcctactcctacatgtacgcgggcttcagctccttcctcattctcgccaccgtcctctgcaacgtgcttgtgtgcggcgcgctgctccgcatgcaccgccagttca

SEQ ID NO: 4 - DNA, 143 bp, synthetic construct
  source 1..143: mol_type = other DNA
ggttgcctcccggggccaccccgctgcctccccagccttgccgcgcctcagcgactttcggcgccgccggagcttccgccgcatcgcgggcgccgagatccagatggtcatcttactcattgccacctccctggtggtgctca

SEQ ID NO: 5 - DNA, 128 bp, synthetic construct
  source 1..128: mol_type = other DNA
ccttgcggtgtgctttctttgcagggcatgcccccgctcagcccggagaagcccgccctgtgcgccggctgcgggggcaagatctcggacaggtactatctgctggctgtggacaaacagtggcatct

SEQ ID NO: 6 - DNA, 190 bp, synthetic construct
  source 1..190: mol_type = other DNA
tccccgcctactccaaggcacagacctgaaatcttctgggaacctggcgggtcgcgacaaggggccaagactcaccttcggggcaccggcaagctacggaacaggtggcggggctgcagcaccccaatgaccgatcaaccgcaaaggccggaaatgcgtcagccgttctgagcccactggctgaagccag

SEQ ID NO: 7 - DNA, 22 bp, synthetic construct
  source 1..22: mol_type = other DNA
tgttgttgatcgtttcgtagcg

SEQ ID NO: 8 - DNA, 23 bp, synthetic construct
  source 1..23: mol_type = other DNA
cgatctatcccctcgaactccta

SEQ ID NO: 9 - DNA, 19 bp, synthetic construct
  source 1..19: mol_type = other DNA
ccgccacccgccgaaaccc

SEQ ID NO: 10 - DNA, 24 bp, synthetic construct
  source 1..24: mol_type = other DNA
gttgttgttgatcgtttcgtagcg

SEQ ID NO: 11 - DNA, 24 bp, synthetic construct
  source 1..24: mol_type = other DNA
gatctatcccctcgaactcctacg

SEQ ID NO: 12 - DNA, 24 bp, synthetic construct
  source 1..24: mol_type = other DNA
ccgaaaccctcctcccgaaacccg

SEQ ID NO: 13 - DNA, 24 bp, synthetic construct
  source 1..24: mol_type = other DNA
gggttgttgttgatcgtttcgtag

SEQ ID NO: 14 - DNA, 24 bp, synthetic construct
  source 1..24: mol_type = other DNA
gatctatcccctcgaactcctacg

SEQ ID NO: 15 - DNA, 25 bp, synthetic construct
  source 1..25: mol_type = other DNA
cgccgaaaccctcctcccgaaaccc

SEQ ID NO: 16 - DNA, 22 bp, synthetic construct
  source 1..22: mol_type = other DNA
tagcgttgtacggtgtacgtcg

SEQ ID NO: 17 - DNA, 23 bp, synthetic construct
  source 1..23: mol_type = other DNA
acaaaacgattacctacccaacg

SEQ ID NO: 18 - DNA, 25 bp, synthetic construct
  source 1..25: mol_type = other DNA
cccgaatactacgccgacgaccaaa

SEQ ID NO: 19 - DNA, 21 bp, synthetic construct
  source 1..21: mol_type = other DNA
ttcgttttcggttggaagttg

SEQ ID NO: 20 - DNA, 18 bp, synthetic construct
  source 1..18: mol_type = other DNA
attacctacccaacgacc

SEQ ID NO: 21 - DNA, 21 bp, synthetic construct
  source 1..21: mol_type = other DNA
cgcccgaatactacgccgacg

SEQ ID NO: 22 - DNA, 24 bp, synthetic construct
  source 1..24: mol_type = other DNA
aagttcgttttcggttggaagttg

SEQ ID NO: 23 - DNA, 24 bp, synthetic construct
  source 1..24: mol_type = other DNA
aaacgattacctacccaacgacca

SEQ ID NO: 24 - DNA, 25 bp, synthetic construct
  source 1..25: mol_type = other DNA
cgcccgaatactacgccgacgacca

SEQ ID NO: 25 - DNA, 25 bp, synthetic construct
  source 1..25: mol_type = other DNA
aatacttcatcgactaaaccaccaa

SEQ ID NO: 26 - DNA, 24 bp, synthetic construct
  source 1..24: mol_type = other DNA
gaatgaggaaggagttgaagttcg

SEQ ID NO: 27 - DNA, 25 bp, synthetic construct
  source 1..25: mol_type = other DNA
taacgacgcacgccgcctactccta

SEQ ID NO: 28 - DNA, 24 bp, synthetic construct
  source 1..24: mol_type = other DNA
tacttcatcgactaaaccaccaac

SEQ ID NO: 29 - DNA, 27 bp, synthetic construct
  source 1..27: mol_type = other DNA
cgtatataagtacgttgtagaggacgg

SEQ ID NO: 30 - DNA, 23 bp, synthetic construct
  source 1..23: mol_type = other DNA
taacgacgcacgccgcctactcc

SEQ ID NO: 31 - DNA, 27 bp, synthetic construct
  source 1..27: mol_type = other DNA
ctaatacttcatcgactaaaccaccaa

SEQ ID NO: 32 - DNA, 24 bp, synthetic construct
  source 1..24: mol_type = other DNA
gaatgaggaaggagttgaagttcg

SEQ ID NO: 33 - DNA, 25 bp, synthetic construct
  source 1..25: mol_type = other DNA
taacgacgcacgccgcctactccta

SEQ ID NO: 34 - DNA, 22 bp, synthetic construct
  source 1..22: mol_type = other DNA
ttagttttgtcgcgttttagcg

SEQ ID NO: 35 - DNA, 30 bp, synthetic construct
  source 1..30: mol_type = other DNA
ataaataaaataaccatctaaatctcgacg

SEQ ID NO: 36 - DNA, 26 bp, synthetic construct
  source 1..26: mol_type = other DNA
cgatacgacgaaaactccgacgacgc

SEQ ID NO: 37 - DNA, 23 bp, synthetic construct
  source 1..23: mol_type = other DNA
ttttagttttgtcgcgttttagc

SEQ ID NO: 38 - DNA, 24 bp, synthetic construct
  source 1..24: mol_type = other DNA
aaataaccatctaaatctcgacgc

SEQ ID NO: 39 - DNA, 23 bp, synthetic construct
  source 1..23: mol_type = other DNA
acgaaaactccgacgacgccgaa

SEQ ID NO: 40 - DNA, 20 bp, synthetic construct
  source 1..20: mol_type = other DNA
attacctcccgaaaccaccc

SEQ ID NO: 41 - DNA, 30 bp, synthetic construct
  source 1..30: mol_type = other DNA
gtaatgagtaagatgattatttggatttcg

SEQ ID NO: 42 - DNA, 25 bp, synthetic construct
  source 1..25: mol_type = other DNA
ccaaccttaccgcgcctcaacgact

SEQ ID NO: 43 - DNA, 26 bp, synthetic construct
  source 1..26: mol_type = other DNA
gtagggtatgttttcgtttagttcgg

SEQ ID NO: 44 - DNA, 26 bp, synthetic construct
  source 1..26: mol_type = other DNA
cacaaccaacaaataatacctatccg

SEQ ID NO: 45 - DNA, 22 bp, synthetic construct
  source 1..22: mol_type = other DNA
cccgcaaccgacgcacaaaacg

SEQ ID NO: 46 - DNA, 24 bp, synthetic construct
  source 1..24: mol_type = other DNA
agggtatgttttcgtttagttcgg

SEQ ID NO: 47 - DNA, 26 bp, synthetic construct
  source 1..26: mol_type = other DNA
cacaaccaacaaataatacctatccg

SEQ ID NO: 48 - DNA, 24 bp, synthetic construct
  source 1..24: mol_type = other DNA
ccccgcaaccgacgcacaaaacga

SEQ ID NO: 49 - DNA, 25 bp, synthetic construct
  source 1..25: mol_type = other DNA
gtagggtatgttttcgtttagttcg

SEQ ID NO: 50 - DNA, 26 bp, synthetic construct
  source 1..26: mol_type = other DNA
caaccaacaaataatacctatccgaa

SEQ ID NO: 51 - DNA, 24 bp, synthetic construct
  source 1..24: mol_type = other DNA
ccccgcaaccgacgcacaaaacga

SEQ ID NO: 52 - DNA, 23 bp, synthetic construct
  source 1..23: mol_type = other DNA
tttcggggtatcggtaagttacg

SEQ ID NO: 53 - DNA, 21 bp, synthetic construct
  source 1..21: mol_type = other DNA
cgcatttccgacctttacgat

SEQ ID NO: 54 - DNA, 33 bp, synthetic construct
  source 1..33: mol_type = other DNA
atcgatcattaaaatactacaaccccgccacct

SEQ ID NO: 55 - DNA, 25 bp, synthetic construct
  source 1..25: mol_type = other DNA
taagatttattttcggggtatcggt

SEQ ID NO: 56 - DNA, 26 bp, synthetic construct
  source 1..26: mol_type = other DNA
accaataaactcaaaacgactaacgc

SEQ ID NO: 57 - DNA, 35 bp, synthetic construct
  source 1..35: mol_type = other DNA
ttccgacctttacgattaatcgatcattaaaatac

SEQ ID NO: 58 - DNA, 30 bp, synthetic construct
  source 1..30: mol_type = other DNA
ttttcgtttattttaaggtatagatttgaa

SEQ ID NO: 59 - DNA, 29 bp, synthetic construct
  source 1..29: mol_type = other DNA
attaatcgatcattaaaatactacaaccc

SEQ ID NO: 60 - DNA, 29 bp, synthetic construct
  source 1..29: mol_type = other DNA
attccgtaacttaccgataccccgaaaat

SEQ ID NO: 61 - DNA, 24 bp, synthetic construct
  source 1..24: mol_type = other DNA
gtgatggaggaggtttagtaagtt

SEQ ID NO: 62 - DNA, 25 bp, synthetic construct
  source 1..25: mol_type = other DNA
ccaataaaacctactcctcccttaa

SEQ ID NO: 63 - DNA, 30 bp, synthetic construct
  source 1..30: mol_type = other DNA
accaccacccaacacacaataacaaacaca

(19) United States
(12) Patent Application Publication
(10) Pub. No.: US 2024/0084393 A1
(43) Pub. Date: Mar. 14, 2024

(54) DNA METHELATION MOLECULAR MARKERS FOR IDENTIFYING BENIGNITY OR MALIGNANCY OF LUNG NODULE AND APPLICATIONS OF THE SAME

(71) Applicant: AnchorDx Medical Co., Ltd., Guangzhou (CN)

(72) Inventors: Zhujia YE, Guangzhou (CN); Hao YANG, Guangzhou (CN); Yanying LIU, Guangzhou (CN); Jinsheng TAO, Guangzhou (CN); Xi LUO, Guangzhou (CN); Jiehan XU, Guangzhou (CN); Zhiwei CHEN, Guangzhou (CN); Jianbing FAN, Guangzhou (CN)

(21) Appl. No.: 18/337,000

(22) Filed: Jun. 17, 2023

Related U.S. Application Data
(63) Continuation-in-part of application No. PCT/CN2021/137231, filed on Dec. 10, 2021.
Application No. PCT/CN2021/137231 is a continuation-in-part of application No. PCT/CN2021/086902, filed on Apr. 13, 2021.

(30) Foreign Application Priority Data
Dec. 17, 2020 (CN) 202011496184.7
Jun. 28, 2022 (CN) 202210753404.2
May 19, 2023 (CN) 202310565785.6

(51) Int. Cl. C12Q 1/6886

(52) U.S. Cl. CPC C12Q 1/6886; C12Q 2600/112; C12Q 2600/154; C12Q 2600/16; C12Q 2600/166

(57) ABSTRACT

A molecular marker for identifying DNA methylation and thereby detecting the benignity or malignancy of a lung nodule is disclosed. The molecular marker for identifying DNA methylation includes the sequence of, or the completely complementary sequence to, SEQ ID NO:6 or a continuous fragment of at least 55% of the full length of the sequence of, or the completely complementary sequence to, SEQ ID NO:6. Also disclosed are applications of the molecular marker for identifying DNA methylation, including a corresponding detection reagent kit and a corresponding detection method. The disclosed molecular marker combinations for identifying DNA methylation are highly correlated to lung cancer, are highly sensitive and specific when used to detect the benignity or malignancy of a lung nodule, and can increase the detection rate of malignant lung nodules while lowering the false positive rate.

### CROSS-REFERENCE TO RELATED APPLICATIONS

This application is a continuation-in-part of International Application No. PCT/CN2021/137231 filed Dec. 10, 2021, which is a continuation-in-part of International Application No. PCT/CN2021/086902 filed Apr. 13, 2021 and claims priority to Chinese Patent Application No. 202011496184.7 filed Dec. 17, 2020; this application also claims priority to Chinese Patent Application No. 202310565785.6 filed May 19, 2023, which claims priority to Chinese Patent Application No. 202210753404.2 filed Jun. 28, 2022. The disclosure of all of the above prior-filed applications is incorporated by reference in its entirety therein.

### INCORPORATION OF SEQUENCE LISTING

This application includes a Sequence Listing which has been submitted in XML format via Patent Center, named “ANDX003US.XML” which is 57 KB in size and created on Jun. 9, 2023. The contents of the Sequence Listing are incorporated herein by reference in their entirety.

### TECHNICAL FIELD

The present invention pertains to the field of biotechnology. More particularly, the invention relates to DNA methylation biomarkers and thereby detecting the benignity or malignancy of a lung nodule, and to applications of the same, wherein the applications include a detection reagent kit and a detection method.

### DESCRIPTION OF RELATED ART

The term “lung nodule,” or “solitary pulmonary nodule,” refers to a high- or low-density solid or sub-solid lesion that is shown in a medical image as a quasi-circular shadow, is solitary, has a clearly defined border and a diameter less than or equal to 3 cm, is surrounded by air-containing lung tissues, and is not accompanied by atelectasis, hilar enlargement, or pleural effusion. Lung nodules tend to invade such organs as the lungs, the hilar lymph nodes in both lungs, the eyes, and the skin, with a chest invasion rate as high as 80%-90%. Many a lung nodule does not exclude the possibility of being an early malignant tumor. Lung nodules are an important indicator of primary lung cancer.

Lung nodules are generally classified as benign or malignant, neither of which, however, has noticeable symptoms. A benign nodule may require a treatment based on its cause, whereas a malignant one calls for an early surgery. The causes of benign lung nodules are often related to autoimmune diseases or various infections, and the causes of malignant lung nodules to lung cancer.

The clinical presentation of lung cancer is rather complicated. The presence or absence, as well as the level of severity and timing of occurrence, of symptoms and signs depends on not only the location and pathological type of the tumor and the presence or absence of metastasis and complications, but also the patient's degree of reaction and tolerance. An early-stage lung cancer typically has vague symptoms or causes no discomfort at all. A central lung cancer, however, has early and relatively severe symptoms. A peripheral lung cancer, on the other hand, has late and relatively mild symptoms, if any. This explains why a lung cancer may have already developed to an advanced stage when clinical symptoms show or when found in a routine examination; early screening, therefore, is of paramount importance. As an early-stage lung cancer tends to manifest itself as a malignant lung nodule, the early screening of lung cancer generally begins with the detection of lung nodules.

In terms of the sampling methods for such detection, a liquid biopsy has such advantages over a tissue biopsy as greater ease of operation, non-invasiveness, high repetitiveness, and allowing a disease to be monitored dynamically. A liquid biopsy for lung cancer detection uses a patient's blood, sputum, or bronchoalveolar lavage (BAL) fluid as a sample and entails detecting and analyzing the DNA of tumor cells and the level of DNA modification, e.g., DNA methylation. As the blood of a patient with lung cancer has a very low ctDNA content, and the ctDNA content differs greatly from one individual to another, one of the major challenges faced by the foregoing detection method is to increase the sensitivity of detection. Sputum is clinically collected by requesting a patient to inhale a nebulized solution and encouraging the patient to cough up the sputum induced by the inhaled solution, and a BAL fluid is clinically collected via a fiberoptic bronchoscope. Sputum and BAL fluids come directly from the lungs and therefore provide higher sensitivity in signal detection than blood samples. As far as the sampling process is concerned, sputum collection is a non-invasive operation safer than the collection of a BAL fluid through a fiberoptic bronchoscope, the latter operation of which involves injecting normal saline into a patient's bronchial alveoli and immediately drawing out the fluid in the alveoli in order to collect a valid body fluid sample from the surfaces of the alveoli, the objective being to examine the compositions of the cells and soluble substances in the sample. BAL is a minimally invasive biopsy method safer than a percutaneous lung biopsy and a surgical biopsy.

The applicant and the inventor of this application have been screening early studies for molecular markers that can be used to identify DNA methylation and thereby detect the benignity or malignancy of a lung nodule effectively. The applicant and the inventor have also searched more advanced studies for molecular markers or combinations thereof that are highly suitable for identifying DNA methylation and thereby detecting the benignity or malignancy of a lung nodule. It is the applicant's and the inventor's goal to be able to detect the benignity or malignancy of a lung nodule in a highly sensitive and specific manner, in particular by performing highly sensitive detection on a respiratory tract fluid sample, so as to provide technical support for non-invasive diagnosis of lung cancer, especially an early-stage lung cancer.

### BRIEF SUMMARY OF THE INVENTION

In view of the above, one objective of the present invention is to provide a molecular marker for identifying DNA methylation and thereby detecting the benignity or malignancy of a lung nodule. It is highly desirable that the molecular marker for identifying DNA methylation has high sensitivity and specificity with regard to the detection of the benignity or malignancy of a lung nodule and can therefore increase the detection rate of malignant lung nodules effectively.

The technical solutions employed to achieve the foregoing objective include the following.

The first aspect of the present invention provides a molecular marker for identifying DNA methylation and thereby detecting the benignity or malignancy of a lung nodule.

The molecular marker for identifying DNA methylation and thereby detecting the benignity or malignancy of a lung nodule includes the sequence of, or the completely complementary sequence to, SEQ ID NO:6 or a continuous fragment of at least 55% of the full length of the sequence of, or the completely complementary sequence to, SEQ ID NO:6.

The second aspect of the present invention provides a use of the molecular marker for identifying DNA methylation and/or a reagent for detecting the methylation level of the molecular marker in preparing a reagent kit for detecting the benignity or malignancy of a lung nodule and/or lung cancer.

The third aspect of the present invention provides a reagent kit for detecting the benignity or malignancy of a lung nodule, wherein the reagent kit includes a reagent for detecting the methylation level of the foregoing molecular marker for identifying DNA methylation.

The fourth aspect of the present invention provides a method for detecting the methylation levels of a combination of the foregoing molecular markers for identifying DNA methylation, wherein the method includes the steps of:
(1) extracting genomic DNA from a test sample;
(2) performing a bisulfite treatment on the extracted genomic DNA to obtain a converted DNA; and
(3) performing multiplex quantitative fluorescent PCR detection on the converted DNA obtained from step (2), using probes designed for the molecular markers for identifying DNA methylation.

The fifth aspect of the present invention provides a method for detecting the benignity or malignancy of a lung nodule, wherein the method includes the steps of:
(1) extracting genomic DNA from a test sample;
(2) performing a bisulfite treatment on the extracted genomic DNA to obtain a converted DNA; and
(3) performing detection with the foregoing reagent kit; the method preferably also including the steps of:
(4) determining whether the sample is valid according to the C_{T} value of a reference gene, and then using the C_{T} value of the reference gene to reconcile the C_{T} value of each molecular marker detected in the valid sample; and
(5) performing a model analysis on the reconciled data, and determining the benignity or malignancy of the lung nodule using the model created.

The inventor has found combinations of DNA methylation-specific molecular markers that are highly correlated to the benignity or malignancy of a lung nodule so that by detecting the methylation levels of those markers, the benignity or malignancy of a lung nodule can be detected more effectively, more sensitively, and more specifically. The invention enhances the sensitivity and specificity of detection of the benignity or malignancy of a lung nodule and can increase the detection rate of early-stage malignant lung nodules effectively, thereby enabling treatments and interventions as early as possible to improve patients' survival rates. In addition, the invention can reduce the false positive rate to prevent over-diagnosis and over-treatment of benign lung nodules. The preferred molecular marker combinations provided by the invention for identifying DNA methylation are highly correlated to the benignity or malignancy of a lung nodule and are particularly suitable for being detected in respiratory tract samples, including respiratory tract fluid samples obtained by a minimally invasive or non-invasive means so that non-invasive detection of the benignity or malignancy of a lung nodule can be achieved.

The present invention also provides primers and probes designed for performing quantitative fluorescent PCR detection on the molecular markers for identifying DNA methylation, wherein the primers can bind to a bisulfite-treated DNA to enable multiplex quantitative fluorescent PCR detection on the converted DNA. Moreover, the reagent kit has been optimized so that multiplex quantitative fluorescent PCR detection can be carried out without interference between the primers and probes designed for different molecular markers for identifying DNA methylation, and this helps increase detection efficiency effectively.

### BRIEF DESCRIPTION OF THE SEVERAL VIEWS OF THE DRAWINGS

FIG. 1 shows the receiver operating characteristic (ROC) curves corresponding to the detection of nodule benignity or malignancy from the 134 lung tissue samples in embodiment 4 using the molecular marker combinations of the present invention for identifying DNA methylation.

FIG. 2 shows the ROC curves corresponding to the detection of nodule benignity or malignancy from the 173 respiratory tract fluid samples in embodiment 5 using the molecular marker combinations of the present invention for identifying DNA methylation.

FIG. 3 shows the ROC curves corresponding to the detection of nodule benignity or malignancy from the 61 respiratory tract fluid samples in embodiment 6 using the molecular marker combinations of the present invention for identifying DNA methylation.

### DETAILED DESCRIPTION OF THE INVENTION

In cases where the conditions of an experiment method used in any of the following embodiments of the present invention are not specified, the conventional conditions, e.g., the conditions stated in Sambrook et al., Molecular Cloning: A Laboratory Manual (Cold Spring Harbor Laboratory Press, New York, 1989), or the conditions suggested by a related manufacturer generally apply. All the common chemical reagents used in the embodiments are commercially available.

Unless otherwise defined, each technical or scientific term used in relation to the present invention has the same connotation as generally understood by a person skilled in the art. All such terms used in this specification serve only to expound the embodiments but not to limit the invention.

As used herein, the terms “a plurality of” and “multiple” refer to two or more than two. The term “and/or” indicates three possible relationships between the objects connected by the term; for example, “A and/or B” may indicate that A exists alone, that A and B coexist, or that B exists alone. The symbol “/” generally indicates “or.”

The present invention provides a molecular marker that can be used to identify DNA methylation and thereby detect the benignity or malignancy of a lung nodule, and that is defined by one or a combination of six methylated areas of four to-be-detected genes, namely HOXB4, PTGER4, LHX9, and ZSCAN31. In some embodiments, the molecular marker for identifying DNA methylation includes the sequence of, or the completely complementary sequence to, SEQ ID NO:6 or a continuous fragment of at least 55% of the full length of the sequence of, or the completely complementary sequence to, SEQ ID NO:6.

In some embodiments, the molecular marker for identifying DNA methylation is a combination of molecular markers that further include at least one selected from the group consisting of the sequences of, or the completely complementary sequences to, SEQ ID NO:1 to SEQ ID NO:5 or at least one selected from the group consisting of continuous fragments each of at least 55% of the full length of one of the sequences of, or the completely complementary sequences to, SEQ ID NO:1 to SEQ ID NO:5.

In some embodiments, the molecular marker for identifying DNA methylation includes the sequences of SEQ ID NO:6 and SEQ ID NO:4 or includes the completely complementary sequences to SEQ ID NO:6 and SEQ ID NO:4.

In some embodiments, the molecular marker for identifying DNA methylation as stated in the last paragraph further includes the sequence of, or the completely complementary sequence to, SEQ ID NO:2; or
includes a combination of continuous fragments each of at least 55% of the full length of one of the sequences of, or the completely complementary sequences to, SEQ ID NO:6, SEQ ID NO:4, and SEQ ID NO:2.

In some embodiments, the molecular marker for identifying DNA methylation includes the sequence of, or the completely complementary sequence to, SEQ ID NO:5 in addition to the sequence of SEQ ID NO:6 or the sequences of SEQ ID NO:6 and SEQ ID NO:4.

In some embodiments, each of the foregoing molecular marker combinations that include the sequence of, or the completely complementary sequence to, SEQ ID NO:5 further includes the sequences of, or the completely complementary sequences to, SEQ ID NO:2 and/or SEQ ID NO:3; or
includes a combination of continuous fragments each of at least 55% of the full length of one of the sequences of, or the completely complementary sequences to, SEQ ID NO:5, SEQ ID NO:6, and SEQ ID NO:2 and/or SEQ ID NO:3; or SEQ ID NO:5, SEQ ID NO:6, SEQ ID NO:4, and SEQ ID NO:2 and/or SEQ ID NO:3.

In some embodiments, each of the molecular marker combinations stated in the paragraph before the last (which include the sequence of, or the completely complementary sequence to, SEQ ID NO:5) further includes the sequences of, or the completely complementary sequences to, SEQ ID NO:1 and/or SEQ ID NO:3; or
includes a combination of continuous fragments each of at least 55% of the full length of one of the sequences of, or the completely complementary sequences to, SEQ ID NO:5, SEQ ID NO:6, and SEQ ID NO:1 and/or SEQ ID NO:3; or SEQ ID NO:5, SEQ ID NO:6, SEQ ID NO:4, and SEQ ID NO:1 and/or SEQ ID NO:3.

In some embodiments, the molecular marker for identifying DNA methylation includes the sequences of, or the completely complementary sequences to, SEQ ID NO:6 and SEQ ID NO:1; or
the molecular marker for identifying DNA methylation includes the sequences of, or the completely complementary sequences to, SEQ ID NO:6 and SEQ ID NO:2; or
the molecular marker for identifying DNA methylation includes the sequences of, or the completely complementary sequences to, SEQ ID NO:6 and SEQ ID NO:3; or
the molecular marker for identifying DNA methylation includes the sequences of, or the completely complementary sequences to, SEQ ID NO:6 and SEQ ID NO:5; or
the molecular marker for identifying DNA methylation includes the sequences of, or the completely complementary sequences to, SEQ ID NO:6, SEQ ID NO:1, and SEQ ID NO:2; or
the molecular marker for identifying DNA methylation includes a combination of continuous fragments each of at least 55% of the full length of one of the sequences of, or the completely complementary sequences to, SEQ ID NO:6 and SEQ ID NO:1; or SEQ ID NO:6 and SEQ ID NO:2; or SEQ ID NO:6 and SEQ ID NO:3; or SEQ ID NO:6 and SEQ ID NO:4; or SEQ ID NO:6, SEQ ID NO:1, and SEQ ID NO:2.

In some embodiments, the molecular marker for identifying DNA methylation includes the sequences of, or the completely complementary sequences to, SEQ ID NO:6, SEQ ID NO:2, and SEQ ID NO:4; or
includes a combination of continuous fragments each of at least 55% of the full length of one of the sequences of, or the completely complementary sequences to, SEQ ID NO:6, SEQ ID NO:2, and SEQ ID NO:4.

In some embodiments, the molecular marker for identifying DNA methylation includes the sequences of, or the completely complementary sequences to, SEQ ID NO:6, SEQ ID NO:1, SEQ ID NO:2, SEQ ID NO:3, and SEQ ID NO:4; or
includes a combination of continuous fragments each of at least 55% of the full length of one of the sequences of, or the completely complementary sequences to, SEQ ID NO:6, SEQ ID NO:1, SEQ ID NO:2, SEQ ID NO:3, and SEQ ID NO:4.

In some embodiments, the molecular marker for identifying DNA methylation includes the sequences of, or the completely complementary sequences to, SEQ ID NO:6, SEQ ID NO:1, SEQ ID NO:3, SEQ ID NO:4, and SEQ ID NO:5; or
includes a combination of continuous fragments each of at least 55% of the full length of one of the sequences of, or the completely complementary sequences to, SEQ ID NO:6, SEQ ID NO:1, SEQ ID NO:3, SEQ ID NO:4, and SEQ ID NO:5.

In some embodiments, the molecular marker for identifying DNA methylation includes the sequences of, or the completely complementary sequences to, SEQ ID NO:1 to SEQ ID NO:6; or includes a combination of continuous fragments each of at least 55% of the full length of one of the sequences of, or the completely complementary sequences to, SEQ ID NO:1 to SEQ ID NO:6.

In some embodiments, the molecular marker for identifying DNA methylation includes the following continuous fragments each of at least 55% of the full length of one of the sequences of SEQ ID NO:1 to SEQ ID NO:6:
the sequence corresponding to a fragment amplified from SEQ ID NO:1 using SEQ ID NO:7 and SEQ ID NO:8, or SEQ ID NO:10 and SEQ ID NO:11, or SEQ ID NO:13 and SEQ ID NO:14 as primers; and/or
the sequence corresponding to a fragment amplified from SEQ ID NO:2 using SEQ ID NO:16 and SEQ ID NO:17, or SEQ ID NO:19 and SEQ ID NO:20, or SEQ ID NO:22 and SEQ ID NO:23 as primers; and/or
the sequence corresponding to a fragment amplified from SEQ ID NO:3 using SEQ ID NO:25 and SEQ ID NO:26, or SEQ ID NO:28 and SEQ ID NO:29, or SEQ ID NO:31 and SEQ ID NO:32 as primers; and/or
the sequence corresponding to a fragment amplified from SEQ ID NO:4 using SEQ ID NO:34 and SEQ ID NO:35, or SEQ ID NO:37 and SEQ ID NO:38, or SEQ ID NO:40 and SEQ ID NO:41 as primers; and/or
the sequence corresponding to a fragment amplified from SEQ ID NO:5 using SEQ ID NO:43 and SEQ ID NO:44, or SEQ ID NO:46 and SEQ ID NO:47, or SEQ ID NO:49 and SEQ ID NO:50 as primers; and/or
the sequence corresponding to a fragment amplified from SEQ ID NO:6 using SEQ ID NO:52 and SEQ ID NO:53, or SEQ ID NO:55 and SEQ ID NO:56, or SEQ ID NO:58 and SEQ ID NO:59 as primers.

In some embodiments, the molecular marker for identifying DNA methylation includes the following continuous fragments each of at least 55% of the full length of one of the sequences of SEQ ID NO:1 to SEQ ID NO:6:
the sequence corresponding to the fragment amplified from SEQ ID NO:1 using SEQ ID NO:7 and SEQ ID NO:8 as primers; and/or
the sequence corresponding to the fragment amplified from SEQ ID NO:2 using SEQ ID NO:16 and SEQ ID NO:17 as primers; and/or
the sequence corresponding to the fragment amplified from SEQ ID NO:3 using SEQ ID NO:28 and SEQ ID NO:29 as primers; and/or
the sequence corresponding to the fragment amplified from SEQ ID NO:4 using SEQ ID NO:37 and SEQ ID NO:38 as primers; and/or
the sequence corresponding to the fragment amplified from SEQ ID NO:5 using SEQ ID NO:43 and SEQ ID NO:44 as primers; and/or
the sequence corresponding to the fragment amplified from SEQ ID NO:6 using SEQ ID NO:52 and SEQ ID NO:53 as primers.

In some embodiments, each of the foregoing molecular marker combinations for identifying DNA methylation is a molecular marker combination to be detected in a respiratory tract sample, preferably a lung tissue sample or a respiratory tract fluid sample.

In some embodiments, the molecular marker for identifying DNA methylation is a combination of at least two selected from the group consisting of the sequences of, or the completely complementary sequences to, SEQ ID NO:1 to SEQ ID NO:6 or is a combination of at least two selected from the group consisting of continuous fragments each of at least 55% of the full length of one of the sequences of, or the completely complementary sequences to, SEQ ID NO:1 to SEQ ID NO:6.

Some embodiments further provide a molecular marker for identifying DNA methylation and thereby detecting the benignity or malignancy of a lung nodule that includes the sequences of, or the completely complementary sequences to, SEQ ID NO:2 and SEQ ID NO:4; or the molecular marker for identifying DNA methylation includes the sequences of, or the completely complementary sequences to, SEQ ID NO:1 to SEQ ID NO:4; or the molecular marker for identifying DNA methylation includes the sequences of, or the completely complementary sequences to, SEQ ID NO:1 to SEQ ID NO:5; or the molecular marker for identifying DNA methylation includes the sequences of, or the completely complementary sequences to, SEQ ID NO:3 and SEQ ID NO:6; or the molecular marker for identifying DNA methylation includes continuous fragments each of at least 55% of the full length of one of the sequences of, or the completely complementary sequences to, SEQ ID NO:2 and SEQ ID NO:4; or SEQ ID NO:1 to SEQ ID NO:4; or SEQ ID NO:1 to SEQ ID NO:5; or SEQ ID NO:3 and SEQ ID NO:6.

In the embodiments described further below, molecular markers for identifying DNA methylation are also referred to as Markers for short, and so are the fragments respectively amplified from those molecular markers with the corresponding primers.

One implementation mode of the present invention uses a combination of at least two of the to-be-detected methylated areas of six molecular markers for identifying DNA methylation and thereby detecting the benignity or malignancy of a lung nodule, and the molecular markers are suitable for being detected in a respiratory tract sample.

One implementation mode of the present invention involves the use of a reagent for detecting a foregoing molecular marker combination for identifying DNA methylation in preparing a reagent kit for detecting the benignity or malignancy of a lung nodule.

One implementation mode of the present invention involves applying a foregoing molecular marker for identifying DNA methylation to the detection of the benignity or malignancy of a lung nodule and/or the detection of lung cancer.

Some implementation modes of the present invention involve a reagent kit for detecting the benignity or malignancy of a lung nodule, and the reagent kit includes a reagent for detecting the methylation level of a foregoing molecular marker for identifying DNA methylation.

In some embodiments, the reagent kit includes a reagent for use in a PCR amplification method, a quantitative fluorescent PCR method, a digital PCR method, a liquid chip method, a first-generation sequencing method, a third-generation sequencing method, a second-generation sequencing method, a pyrosequencing method, a bisulfite conversion-based sequencing method, a methylation chip method, a reduced-representation bisulfite sequencing method, or a combination of the above. The present invention has no limitation on the method to be used by the reagent, provided that the method furnishes a platform capable of high-throughput detection.

In some embodiments, the reagent includes the following combinations of primers and probe for performing quantitative fluorescent PCR detection on the corresponding molecular marker(s) for identifying DNA methylation:
for SEQ ID NO:1, at least one combination selected from the group consisting of: primers with the sequences of SEQ ID NO:7 and SEQ ID NO:8 respectively and a probe with the sequence of SEQ ID NO:9; primers with the sequences of SEQ ID NO:10 and SEQ ID NO:11 respectively and a probe with the sequence of SEQ ID NO:12; and primers with the sequences of SEQ ID NO:13 and SEQ ID NO:14 respectively and a probe with the sequence of SEQ ID NO:15; and/or
for SEQ ID NO:2, at least one combination selected from the group consisting of: primers with the sequences of SEQ ID NO:16 and SEQ ID NO:17 respectively and a probe with the sequence of SEQ ID NO:18; primers with the sequences of SEQ ID NO:19 and SEQ ID NO:20 respectively and a probe with the sequence of SEQ ID NO:21; and primers with the sequences of SEQ ID NO: 22 and SEQ ID NO:23 respectively and a probe with the sequence of SEQ ID NO:24; and/or
for SEQ ID NO:3, at least one combination selected from the group consisting of: primers with the sequences of SEQ ID NO:25 and SEQ ID NO:26 respectively and a probe with the sequence of SEQ ID NO:27; primers with the sequences of SEQ ID NO:28 and SEQ ID NO:29 respectively and a probe with the sequence of SEQ ID NO:30; and primers with the sequences of SEQ ID NO:31 and SEQ ID NO:32 respectively and a probe with the sequence of SEQ ID NO:33; and/or
for SEQ ID NO:4, at least one combination selected from the group consisting of: primers with the sequences of SEQ ID NO:34 and SEQ ID NO:35 respectively and a probe with the sequence of SEQ ID NO:36; primers with the sequences of SEQ ID NO:37 and SEQ ID NO:38 respectively and a probe with the sequence of SEQ ID NO:39; and primers with the sequences of SEQ ID NO:40 and SEQ ID NO:41 respectively and a probe with the sequence of SEQ ID NO:42; and/or
for SEQ ID NO:5, at least one combination selected from the group consisting of: primers with the sequences of SEQ ID NO:43 and SEQ ID NO:44 respectively and a probe with the sequence of SEQ ID NO:45; primers with the sequences of SEQ ID NO:46 and SEQ ID NO:47 respectively and a probe with the sequence of SEQ ID NO:48; and primers with the sequences of SEQ ID NO:49 and SEQ ID NO:50 respectively and a probe with the sequence of SEQ ID NO:51; and/or
for SEQ ID NO:6, at least one combination selected from the group consisting of: primers with the sequences of SEQ ID NO:52 and SEQ ID NO:53 respectively and a probe with the sequence of SEQ ID NO:54; primers with the sequences of SEQ ID NO:55 and SEQ ID NO:56 respectively and a probe with the sequence of SEQ ID NO:57; and primers with the sequences of SEQ ID NO:58 and SEQ ID NO:59 respectively and a probe with the sequence of SEQ ID NO:60; or
at least one combination of primers and probe each having a plurality of continuous nucleotides with at least 70%, 80%, 90%, 95%, or 99% sequence identity to the sequence of one or the other primer or the probe in any of the foregoing combinations.

In some embodiments, the reagent includes the following combinations of primers and probe:
for SEQ ID NO:1, the primers with the sequences of SEQ ID NO:7 and SEQ ID NO:8 respectively and the probe with the sequence of SEQ ID NO:9; and/or
for SEQ ID NO:2, the primers with the sequences of SEQ ID NO:16 and SEQ ID NO:17 respectively and the probe with the sequence of SEQ ID NO:18; and/or
for SEQ ID NO:3, the primers with the sequences of SEQ ID NO:28 and SEQ ID NO:29 respectively and the probe with the sequence of SEQ ID NO:30; and/or
for SEQ ID NO:4, the primers with the sequences of SEQ ID NO:37 and SEQ ID NO:38 respectively and the probe with the sequence of SEQ ID NO:39; and/or
for SEQ ID NO:5, the primers with the sequences of SEQ ID NO:43 and SEQ ID NO:44 respectively and the probe with the sequence of SEQ ID NO:45; and/or
for SEQ ID NO:6, the primers with the sequences of SEQ ID NO:52 and SEQ ID NO:53 respectively and the probe with the sequence of SEQ ID NO:54.

In some embodiments, the reagent kit further includes primers and a probe for performing quantitative fluorescent PCR detection on a reference gene ACTB. More specifically, the primers and the probe for the reference gene ACTB are primers with the sequences of SEQ ID NO:61 and SEQ ID NO:62 respectively and a probe with the sequence of SEQ ID NO:63.

For the present invention, primer and probe design was carried out for each marker in the foregoing molecular marker combinations for identifying DNA methylation, or more particularly for identifying the methylated areas specified herein. The amplification primers obtained for the molecular marker combinations for identifying DNA methylation have been applied to genomic DNA (gDNA) that is extracted from respiratory tract samples and treated with bisulfite, and multiplex quantitative fluorescent PCR detection has been performed to detect the methylation signals of the to-be-detected areas of the gDNA with the probes, which are respectively specific to the molecular markers for identifying DNA methylation. A benignity/malignancy prediction model has also been created with the naive Bayes algorithms and then used to diagnose the benignity or malignancy of lung nodules.

Some embodiments of the present invention further provide a method for detecting the methylation levels of a molecular marker combination for identifying DNA methylation, and the method includes the steps of:
(1) extracting genomic DNA from a test sample;
(2) performing a bisulfite treatment on the extracted genomic DNA to obtain a converted DNA; and
(3) performing multiplex quantitative fluorescent PCR detection on the converted DNA obtained from step (2), using the probes designed specifically for the molecular markers for identifying DNA methylation.

In some embodiments, the multiplex quantitative fluorescent PCR takes place under the following reaction conditions:
polymerase activation: 95° C., 5 min;
amplification I: 95° C., 15 s; 10-20 cycles, 60° C-66° C., 30 s; and
amplification II: 95° C., 15 s; 40-60 cycles, 60° C-64° C., 30 s.

In some embodiments, determining the sample as a valid sample if the reference gene has a C_{T} value of 10-25 or as an invalid sample if otherwise; reconciling the C_{T} value of each molecular marker in the valid sample according to the C_{T} value of the reference gene; and determining that a target molecular marker for identifying DNA methylation has been detected, and obtaining a relative cycle number ΔC_{T} of the target molecular marker for identifying DNA methylation, if the C_{T} value of the target molecular marker for identifying DNA methylation is less than 50, wherein ΔC_{T}=the C_{T} value of the target molecular marker for identifying DNA methylation−the C_{T} value of the reference gene; or determining that the target molecular marker for identifying DNA methylation has not been detected, and assigning ΔC_{T}=40 to the target molecular marker for identifying DNA methylation, if the C_{T} value of the target molecular marker for identifying DNA methylation is “undetermined”.

In some embodiments, performing a data analysis on the reconciled ΔC_{T} values, and creating a lung nodule benignity/malignancy prediction model by a logistic regression calculation method. During the modeling process, a cross-validation method is used to randomly divide the data set into three equal parts and then combine each two of those parts into a training set while the remaining part serves as a test set. Based on the principle of combination, three different training and test set combinations can be obtained from the three randomly divided equal parts. The logistic regression calculation method is then used to create a benignity/malignancy prediction model out of the different molecular marker combinations for identifying DNA methylation in the training sets, and the classification ability of the model is evaluated with the training sets including the molecular marker combination of interest. 100 random and mutually independent tests are conducted according to the foregoing steps, and the classification ability of the final model for the molecular marker combination of interest is determined as the average classification ability of 100 models.

The present invention is explained in more detail below by way of specific embodiments, which, however, are not intended to limit the scope of patent protection sought by the applicant.

### Embodiment 1

In this and the following embodiments, Marker1 to Marker6 refer to molecular markers that are defined respectively by six methylated areas of four to-be-detected genes, namely HOXB4, PTGER4, LHX9, and ZSCAN31, and that are provided by the present invention for identifying DNA methylation and thereby detecting the benignity or malignancy of a lung nodule. This embodiment provides a reagent kit that includes detection primers and probes designed specifically for those Markers. Table 1 shows the sequence of the to-be-detected area, as well as the sequence ID number, of each of the molecular markers for identifying DNA methylation (with the underlined portion of each Marker being the sequence corresponding to the fragment to be amplified with the corresponding preferred primers in the following embodiments).

TABLE 1
Sequences of the to-be-detected areas of six molecular markers

| Name of to- | Name of | | |
|---|---|---|---|
| be-detected | molecular | SEQ | Sequence |
| gene | marker | ID NO: | of the to-be-detected area of the molecular marker |
| HOXB4 | Marker1 | 1 | GTTCTGGGCGCAGGGAGGCGGCGGGGGGCTGCTGCTGACCGC |
| | | | CTCGCAGCGCTGGCCGGGCTCCGGGAGGAGGGCCCCGGCGGG |
| | | | TGGCGGCGCAGGAGCCCGAGGGGACAGACCGGGCGGTGGCG |
| | | | GGGGCGGCGGGGGTGGTGGC |
| | Marker2 | 2 | GCGGGGGCCCAGGGTCCCGGCAGGCCGCGTAGCGCTGCACGG |
| | | | TGCACGCCGCGCGCCGCCCGAAGCCCGCCTCCGGCTGGAAGC |
| | | | TGCTCTCTCGCCTCTGGCCGCCGGCGTAGTACCCGGGCGAGTG |
| | | | GTCGCTGGGTAGGTAATCGCTCTGTGAATATTCCTCGCATGGAG |
| | | | GGAACTTGGGG |
| PTGER4 | Marker3 | 3 | GCTCGCGGCTGCAGTACCCAGACACCTGGTGCTTCATCGACTG |
| | | | GACCACCAACGTGACGGCGCACGCCGCCTACTCCTACATGTAC |
| | | | GCGGGCTTCAGCTCCTTCCTCATTCTCGCCACCGTCCTCTGCA |
| | | | ACGTGCTTGTGTGCGGCGCGCTGCTCCGCATGCACCGCCAGTT |
| | | | CA |
| | Marker4 | 4 | GGTTGCCTCCCGGGGCCACCCCGCTGCCTCCCCAGCCTTGCCG |
| | | | CGCCTCAGCGACTTTCGGCGCCGCCGGAGCTTCCGCCGCATCG |
| | | | CGGGCGCCGAGATCCAGATGGTCATCTTACTCATTGCCACCTC |
| | | | CCTGGTGGTGCTCA |
| LHX9 | Marker5 | 5 | CCTTGCGGTGTGCTTTCTTTGCAGGGCATGCCCCCGCTCAGCC |
| | | | CGGAGAAGCCCGCCCTGTGCGCCGGCTGCGGGGGCAAGATCT |
| | | | CGGACAGGTACTATCTGCTGGCTGTGGACAAACAGTGGCATCT |
| ZSCAN31 | Marker6 | 6 | TCCCCGCCTACTCCAAGGCACAGACCTGAAATCTTCTGGGAAC |
| | | | CTGGCGGGTCGCGACAAGGGGCCAAGACTCACCTTCGGGGCA |
| | | | CCGGCAAGCTACGGAACAGGTGGCGGGGCTGCAGCACCCCAA |
| | | | TGACCGATCAACCGCAAAGGCCGGAAATGCGTCAGCCGTTCT |
| | | | GAGCCCACTGGCTGAAGCCAG |

For the reagent kit in this embodiment, three pairs of primers and three probes were designed for the specific methylated site of each of the six molecular markers, i.e., Marker 1 to Marker6, to be detected in respiratory tract samples in order to identify the benignity or malignancy of a lung nodule. (The probes, or fluorescent markers, may be any fluorescent-group marker such as FAM, VIC, or NED.) The three pairs of primers and the three probes for each Marker were divided into three groups, namely combinations 1, 2, and 3. The primer and probe combination 1, 2, or 3 chosen for a certain molecular marker can be combined with the primer and probe combination 1, 2, or 3 chosen for another molecular marker in order to be detected on the same platform. The primer and probe sequences corresponding to each molecular marker are shown in Table 2.

TABLE 2
Primer and probe sequences corresponding to each molecular marker

| SEQ ID NO: | | | | | | | |
|---|---|---|---|---|---|---|---|
| corresponding | | SEQ | | SEQ | | SEQ | |
| to to-be- | Comb. | ID | Forward | ID | Reverse | ID | |
| detected area | NO. | NO: | primer | NO: | primer | NO: | Probe |
| 1 | 1 | 7 | TGTTGTTGATCG | 8 | CGATCTATCCCC | 9 | CCGCCACCCGC |
| | | | TTTCGTAGCG | | TCGAACTCCTA | | CGAAACCC |
| | 2 | 10 | GTTGTTGTTGAT | 11 | GATCTATCCCCT | 12 | CCGAAACCCTC |
| | | | CGTTTCGTAGC | | CGAACTCCTAC | | CTCCCGAAACC |
| | | | G | | G | | CG |
| | 3 | 13 | GGGTTGTTGTT | 14 | GATCTATCCCCT | 15 | CGCCGAAACCC |
| | | | GATCGTTTCGTA | | CGAACTCCTAC | | TCCTCCCGAAA |
| | | | G | | G | | CCC |
| 2 | 1 | 16 | TAGCGTTGTACG | 17 | ACAAAACGATT | 18 | CCCGAATACTAC |
| | | | GTGTACGTCG | | ACCTACCCAAC | | GCCGACGACCA |
| | | | | | G | | AA |
| | 2 | 19 | TTCGTTTTCGGT | 20 | ATTACCTACCCA | 21 | CGCCCGAATAC |
| | | | TGGAAGTTG | | ACGACC | | TACGCCGACG |
| | 3 | 22 | AAGTTCGTTTTC | 23 | AAACGATTACCT | 24 | CGCCCGAATAC |
| | | | GGTTGGAAGTT | | ACCCAACGACC | | TACGCCGACGA |
| | | | G | | A | | CCA |
| 3 | 1 | 25 | AATACTTCATCG | 26 | GAATGAGGAAG | 27 | TAACGACGCAC |
| | | | ACTAAACCACC | | GAGTTGAAGTT | | GCCGCCTACTC |
| | | | AA | | CG | | CTA |
| | 2 | 28 | TACTTCATCGAC | 29 | CGTATATAAGTA | 30 | TAACGACGCAC |
| | | | TAAACCACCAA | | CGTTGTAGAGG | | GCCGCCTACTC |
| | | | C | | ACGG | | C |
| | 3 | 31 | CTAATACTTCAT | 32 | GAATGAGGAAG | 33 | TAACGACGCAC |
| | | | CGACTAAACCA | | GAGTTGAAGTT | | GCCGCCTACTC |
| | | | CCAA | | CG | | CTA |
| 4 | 1 | 34 | TTAGTTTTGTCG | 35 | ATAAATAAAATA | 36 | CGATACGACGA |
| | | | CGTTTTAGCG | | ACCATCTAAATC | | AAACTCCGACG |
| | | | | | TCGACG | | ACGC |
| | 2 | 37 | TTTTAGTTTTGT | 38 | AAATAACCATCT | 39 | ACGAAAACTCC |
| | | | CGCGTTTTAGC | | AAATCTCGACG | | GACGACGCCGA |
| | | | | | C | | A |
| | 3 | 40 | ATTACCTCCCGA | 41 | GTAATGAGTAA | 42 | CCAACCTTACC |
| | | | AACCACCC | | GATGATTATTTG | | GCGCCTCAACG |
| | | | | | GATTTCG | | ACT |
| 5 | 1 | 43 | GTAGGGTATGTT | 44 | CACAACCAACA | 45 | CCCGCAACCGA |
| | | | TTCGTTTAGTTC | | AATAATACCTAT | | CGCACAAAACG |
| | | | GG | | CCG | | |
| | 2 | 46 | AGGGTATGTTTT | 47 | CACAACCAACA | 48 | CCCCGCAACCG |
| | | | CGTTTAGTTCGG | | AATAATACCTAT | | ACGCACAAAAC |
| | | | | | CCG | | GA |
| | 3 | 49 | GTAGGGTATGTT | 50 | CAACCAACAAA | 51 | CCCCGCAACCG |
| | | | TTCGTTTAGTTC | | TAATACCTATCC | | ACGCACAAAAC |
| | | | G | | GAA | | GA |
| 6 | 1 | 52 | TTTCGGGGTATC | 53 | CGCATTTCCGAC | 54 | ATCGATCATTAA |
| | | | GGTAAGTTACG | | CTTTACGAT | | AATACTACAACC |
| | | | | | | | CCGCCACCT |
| | 2 | 55 | TAAGATTTATTT | 56 | ACCAATAAACT | 57 | TTCCGACCTTTA |
| | | | TCGGGGTATCG | | CAAAACGACTA | | CGATTAATCGAT |
| | | | GT | | ACGC | | CATTAAAATAC |
| | 3 | 58 | TTTTCGTTTATT | 59 | ATTAATCGATCA | 60 | ATTCCGTAACTT |
| | | | TTAAGGTATAGA | | TTAAAATACTAC | | ACCGATACCCC |
| | | | TTTGAA | | AACCC | | GAAAAT |

The preferred primer and probe combinations in this and the following embodiments are as follows: primer and probe combination 1 for Marker1, primer and probe combination 1 for Marker2, primer and probe combination 2 for Marker3, primer and probe combination 2 for Marker4, primer and probe combination 1 for Marker5, and primer and probe combination 1 for Marker6.

The reagent kit further includes the primers and probe for a reference gene ACTB, with the primer and probe sequences shown in Table 3.

TABLE 3
Primers and probe for reference gene ACTB

| SEQ | Forward primer | SEQ | Reverse primer | SEQ | |
|---|---|---|---|---|---|
| ID NO: | sequence | ID NO: | sequence | ID NO: | Probe sequence |
| 61 | GTGATGGAGGAG | 62 | CCAATAAAACCTAC | 63 | ACCACCACCCAACAC |
| | GTTTAGTAAGTT | | TCCTCCCTTAA | | ACAATAACAAACACA |

### Embodiment 2

In this embodiment, the reagent kit in embodiment 1 was used to detect the methylation levels of Marker1 to Marker6 in respiratory tract samples.

The method used in this embodiment to detect the methylation level of a molecular marker for identifying DNA methylation was carried out in the following steps:
1. Extracting gDNA from a respiratory tract sample:
   1) To extract gDNA from the respiratory tract sample, the respiratory tract sample was centrifuged at a low speed (5000×g) at 4° C. for 5 min, and then the precipitate was collected by removing the supernatant. The extraction of gDNA was performed according to the user manual of Qiagen's DNeasy® Blood & Tissue Kit.
   2) To extract gDNA from a paraffin-embedded lung tissue sample section instead, please refer to the user manual of Qiagen's ALLPrep DNA/RNA FFPE Kit, for example, for the steps of extracting gDNA from a paraffin-embedded tissue sample.
2. Performing bisulfite conversion on the extracted gDNA:

To perform bisulfite conversion on the extracted gDNA, 40-80 ng of the gDNA (preferably 50 ng as in this embodiment) was used in accordance with the user manual of Zymo's DNA Methylation-Direct MagPrep such that the unmethylated cytosine in the DNA was converted into uracil by deamination while the methylated cytosine remained unchanged.
3. Performing multiplex quantitative fluorescent PCR detection on the conversion product:

All the bisulfite-converted DNA was used in multiplex quantitative fluorescent PCR. The reaction components of the multiplex quantitative fluorescent PCR included a primer-probe mixed solution in which the concentration of each primer was 100-500 nM, preferably 200 nM as in this embodiment, and in which the concentration of the probe was 50-150 nM, preferably 100 nM as in this embodiment. The Methy Tect Taq HS PCR reagent kit (Accurate Biology, Cat#AG11209) was used, and each reaction was a 25 μL system.

The reaction conditions were as follows: pre-denaturation at 95° C. for 5 min; amplification I: 95° C., 15 s; 10-20 cycles, annealing at 60° C.-66° C., 30 s (preferably 15 cycles and 65° C. as in this embodiment); amplification II: 95° C., 15 s; 40-60 cycles, annealing at 60° C.-64° C., 30 s (preferably 50 cycles and 62° C. as in this embodiment). Table 5 shows the design of the fluorescent qPCR system.

TABLE 5
The fluorescent qPCR system

| Component | Volume (μL) | Final concentration | |
|---|---|---|---|
| 10 × Methy Tect PCR | 2.5 | 1× | |
| Buffer (Mg²⁺ free) | | | |
| MgCl₂ (50 mM) | 1.2 | 2.4 | mM |
| dNTPs (10 mM) | 1 | 0.4 | mM |
| TE buffer | 6.605 | — | |
| Primer-probe mixed | 3 | (forward/reverse | |
| solution (75 μM primer, | | primer: each at 200 nM; | |
| 10 μM probe) | | probe: 100 nM) | |
| 25 μM ROX | 0.045 | 0.045 | μM |
| 0.1% Tween20 | 0.05 | 0.002‰ | |
| DNA template | 10 | — | |
| Total volume | 25 μL | | |

The method used in this embodiment to detect the benignity or malignancy of a lung nodule was carried out in the following steps:
4. Determining the validity of the test sample according to the quantitative fluorescent PCR detection result of the C_{T} value of the reference gene in the sample; or more specifically, determining the test sample as a valid sample if the C_{T} value of the reference gene in the sample ranges from 10 to 25, and excluding from further detection and analysis an initially introduced sample that is determined to be an invalid sample;
5. In the case that the sample is determined as a valid sample, determining that a target molecular marker for identifying DNA methylation has been detected, and obtaining a relative cycle number ΔC_{T} of the target molecular marker for identifying DNA methylation, if the C_{T} value of the target molecular marker for identifying DNA methylation is less than 50, wherein ΔC_{T}=the C_{T} value of the target molecular marker for identifying DNA methylation−the C_{T} value of the reference gene; or determining that the target molecular marker for identifying DNA methylation has not been detected, and assigning ΔC_{T}=40 to the target molecular marker for identifying DNA methylation, if the C_{T} value of the target molecular marker for identifying DNA methylation is “undetermined”; and
6. Performing a data analysis on the reconciled ΔC_{T} values, and creating a lung nodule benignity/malignancy prediction model by a logistic regression calculation method, or more specifically, performing a modeling process in which a cross-validation method is used to randomly divide the data set into three equal parts and then combine each two of those parts into a training set while the remaining part serves as a test set (based on the principle of combination, three different training and test set combinations can be obtained from the three randomly divided equal parts); in which the logistic regression calculation method is used to create a benignity/malignancy prediction model out of the different molecular marker combinations for identifying DNA methylation in the training sets, and the classification ability of the model is evaluated with the training sets including the molecular marker combination of interest; and in which 100 random and mutually independent tests are conducted according to the foregoing steps, and the classification ability of the final model for the molecular marker combination of interest is determined as the average classification ability of 100 models.

### Embodiment 3

The method used in this embodiment to detect a molecular marker in the corresponding standards was carried out in the following steps:
1. Preparing the standards:
   1) Preparing a 0% methylation standard:
The 0% methylation standard was prepared by treating NA12878 DNA with REPLI-g® Single Cell Kit (Qiagen, Cat# 150343) and Mung Bean Nuclease (NEB, Cat# M0250L).
   2) Preparing a 100% methylation standard:

The 100% methylation standard was prepared by treating the 0% methylation standard with CpG methyltransferase (M.SssI).
2. Preparing standards of different methylation percentages:
   0.2%, 0.4%, and 1% methylation standards were prepared by mixing the 0% and 100% methylation standards according to the desired methylation percentage gradient.
3. Performing bisulfite conversion on the DNA standards of different methylation percentages:
   40-80 ng of each standard (preferably 50 ng as in this embodiment) was subjected to the conversion, as stated in relation to the corresponding step in embodiment 2.
4. Performing quantitative fluorescent PCR detection on each converted DNA standard as stated in relation to the corresponding step in embodiment 2;
5. Determining the validity of each test sample according to the quantitative fluorescent PCR detection result of the C_{T} value of the reference gene in the sample; or more specifically, determining each test sample as a valid sample if the C_{T} value of the reference gene in the sample ranges from 10 to 25; and
6. In the case that a sample in question is determined as a valid sample, determining that a target molecular marker for identifying DNA methylation has been detected if the C_{T} value of the target molecular marker for identifying DNA methylation is less than 50, or determining that the target molecular marker for identifying DNA methylation has not been detected if the C_{T} value of the target molecular marker for identifying DNA methylation is “undetermined.”

In this and the following embodiments, the primer and probe combination used for each molecular marker was the corresponding preferred combination in embodiment 1.

In this and the following embodiments, each test included a negative control, which used water as the template and was subjected to the same quantitative fluorescent PCR detection as the corresponding molecular marker. If the negative control of a test produced no detection signal, then it was determined that there was no external contamination during the conduction of the entire test.

In this embodiment, three totally independent duplicate tests were conducted for each standard. All the six molecular markers produced detection signals in their respective 100% methylation standards, and none of the molecular markers produced detection signals in their respective negative controls or non-methylated standards. In particular, each of Marker5 and Marker6 produced detection signals in all the three tests for each standard of a methylation percentage≥0.2%; in other words, these two molecular markers had a detection rate as high as 100% in samples with a methylation percentage≥0.2%. In addition, each of the six molecular markers produced detection signals in all the three tests for the corresponding 1% methylation standard; that is to say, all the molecular markers produced detection signals when 1% methylated.

The test results are summarized in Table 6.

TABLE 6
Test results of six molecular markers in standards
of different methylation percentages

| | Number of times of being detected in | | |
|---|---|---|---|
| Name of | three duplicate tests | | |
| molecular | 0.2% | 0.4% | 1% |
| marker | methylation | methylation | methylation |
| Marker1 | 2 | 2 | 3 |
| Marker2 | 1 | 1 | 3 |
| Marker3 | 2 | 3 | 3 |
| Marker4 | 2 | 2 | 3 |
| Marker5 | 3 | 3 | 3 |
| Marker6 | 3 | 3 | 3 |
| Reference gene | 3 | 3 | 3 |

### Embodiment 4: Detection Performances of Six Molecular Markers in Benign or Malignant Lung Nodule Tissue Samples

In this embodiment, the foregoing six molecular markers in 134 lung tissue samples were detected individually. 57 of the samples had been determined as benign by surgical biopsy, and the remaining 77 samples malignant. The malignant samples included 66 stage-I samples, four stage-II samples, and five stage-III samples. The detection reagent kit, detection method, and data analysis criteria used in this embodiment were the same as those in embodiment 2. The primer and probe combinations employed were the preferred combinations in embodiment 1.

Single-molecular-marker detection was performed on Marker1, Marker2, Marker3, Marker4, Marker5, and Marker6 in the lung tissue samples. All the six molecular markers are highly correlated to lung cancer. The performance of each Marker's being individually detected in a lung tissue sample is summarized in Table 7. The AUC (area under the ROC curve) value corresponding to each individual Marker ranged from 0.77 to 0.92. For more detailed detection results, please refer to Table 7.

TABLE 7

| Marker | | 95% CI | | Youden Cutoff | |
|---|---|---|---|---|---|
| ID | AUC | AUC.lower | AUC.upper | Sensitivity | Specificity |
| Marker1 | 0.78 | 0.71 | 0.86 | 0.57 | 0.93 |
| Marker2 | 0.82 | 0.74 | 0.89 | 0.75 | 0.70 |
| Marker3 | 0.88 | 0.82 | 0.94 | 0.90 | 0.39 |
| Marker4 | 0.90 | 0.85 | 0.95 | 0.84 | 0.82 |
| Marker5 | 0.77 | 0.69 | 0.85 | 0.86 | 0.32 |
| Marker6 | 0.82 | 0.75 | 0.89 | 0.75 | 0.88 |

In this embodiment, detection of different combinations of the six molecular markers was also performed. The samples and detection method employed were the same as stated above.

The combinations of the molecular markers detected are detailed in Table 8.

TABLE 8
Different molecular marker combinations

| | Marker1 | Marker2 | Marker3 | Marker4 | Marker5 | Marker6 |
|---|---|---|---|---|---|---|
| Combination 1 | ✓ | ✓ | ✓ | ✓ | ✓ | ✓ |
| Combination 2 | | | ✓ | ✓ | ✓ | ✓ |
| Combination 3 | ✓ | | ✓ | | ✓ | ✓ |
| Combination 4 | ✓ | ✓ | ✓ | ✓ | | |
| Combination 5 | | ✓ | ✓ | ✓ | ✓ | ✓ |
| Combination 6 | ✓ | | ✓ | ✓ | ✓ | ✓ |
| Combination 7 | ✓ | ✓ | ✓ | ✓ | ✓ | |
| Combination 8 | ✓ | ✓ | ✓ | ✓ | | ✓ |
| Combination 9 | | | | ✓ | ✓ | ✓ |
| Combination 10 | | | | ✓ | | ✓ |
| Combination 11 | | | ✓ | | | ✓ |
| Combination 12 | | ✓ | | | | ✓ |
| Combination 13 | ✓ | | | | | ✓ |
| Combination 14 | | | | | ✓ | ✓ |
| Combination 15 | | ✓ | | ✓ | | |
| Combination 16 | ✓ | ✓ | | | | ✓ |
| Combination 17 | | ✓ | | ✓ | | ✓ |

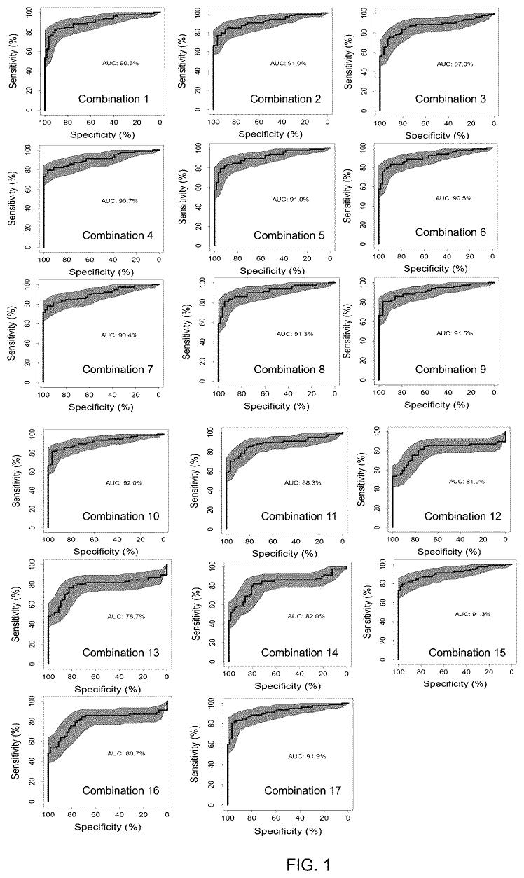

A modeling analysis was performed based on lung nodule tissue samples in which molecular marker combination 1 was chosen as the detection target. According to the analysis results, the model created had an average AUC of 0.91 (specificity: 91%; sensitivity: 82%) and had 82% sensitivity to stage-I malignant samples, 100% sensitivity to stage-II samples, and 100% sensitivity to stage-III samples. The corresponding ROC curve is shown in FIG. 1.

Another modeling analysis was performed based on lung nodule tissue samples in which molecular marker combination 2 was chosen as the detection target. According to the analysis results, the model created had an average AUC of 0.91 (specificity: 96%; sensitivity: 75%) and had 74% sensitivity to stage-I malignant samples, 100% sensitivity to stage-II samples, 100% sensitivity to stage-III samples. The corresponding ROC curve is shown in FIG. 1.

Another modeling analysis was performed based on lung nodule tissue samples in which molecular marker combination 3 was chosen as the detection target. According to the analysis results, the model created had an average AUC of 0.87 (specificity: 91%; sensitivity: 75%) and had 74% sensitivity to stage-I malignant samples, 100% sensitivity to stage-II samples, and 100% sensitivity to stage-III samples. The corresponding ROC curve is shown in FIG. 1.

Another modeling analysis was performed based on lung nodule tissue samples in which molecular marker combination 4 was chosen as the detection target. According to the analysis results, the model created had an average AUC of 0.91 (specificity: 96%; sensitivity: 79%) and had 79% sensitivity to stage-I malignant samples, 100% sensitivity to stage-II samples, and 100% sensitivity to stage-III samples. The corresponding ROC curve is shown in FIG. 1.

Another modeling analysis was performed based on lung nodule tissue samples in which molecular marker combination 5 was chosen as the detection target. According to the analysis results, the model created had an average AUC of 0.91 (specificity: 91%; sensitivity: 82%) and had 82% sensitivity to stage-I malignant samples, 100% sensitivity to stage-II samples, and 100% sensitivity to stage-III samples. The corresponding ROC curve is shown in FIG. 1. Combination 5 had similar performance to combination 1 but lacked Marker1 in combination 1, so it can be inferred that the five molecular markers in combination 5 can work just as well as the six molecular markers in combination 1.

Another modeling analysis was performed based on lung nodule tissue samples in which molecular marker combination 6 was chosen as the detection target. According to the analysis results, the model created had an average AUC of 0.91 (specificity: 96%; sensitivity: 75%) and had 74% sensitivity to stage-I malignant samples, 100% sensitivity to stage-II samples, and 100% sensitivity to stage-III samples. The corresponding ROC curve is shown in FIG. 1. Combination 6 was different from combination 1 only in that the former lacked Marker2, and combination 1, which included Marker2, had better performance than combination 6, which did not include Marker2.

Another modeling analysis was performed based on lung nodule tissue samples in which molecular marker combination 7 was chosen as the detection target. According to the analysis results, the model created had an average AUC of 0.90 (specificity: 96%; sensitivity: 78%) and had 77% sensitivity to stage-I malignant samples, 100% sensitivity to stage-II samples, and 100% sensitivity to stage-III samples. The corresponding ROC curve is shown in FIG. 1. Combination 7 was different from combination 1 only in that the former lacked Marker6, and combination 1, which included Marker6, had better performance than combination 7, which did not include Marker6.

Another modeling analysis was performed based on lung nodule tissue samples in which molecular marker combination 8 was chosen as the detection target. According to the analysis results, the model created had an average AUC of 0.91 (specificity: 95%; sensitivity: 81%) and had 80% sensitivity to stage-I malignant samples, 100% sensitivity to stage-II samples, and 100% sensitivity to stage-III samples. The corresponding ROC curve is shown in FIG. 1. Combination 8 was different from combination 1 only in that the former lacked Marker5, and although the two combinations had similar performances in general, combination 1, which included Marker5, had slightly higher sensitivity to stage-I malignant samples than combination 8, which did not include Marker5.

Another modeling analysis was performed based on lung nodule tissue samples in which molecular marker combination 9 was chosen as the detection target. According to the analysis results, the model created had an average AUC of 0.92 (specificity: 96%; sensitivity: 81%) and had 80% sensitivity to stage-I malignant samples, 100% sensitivity to stage-II samples, and 100% sensitivity to stage-III samples.

Another modeling analysis was performed based on lung nodule tissue samples in which molecular marker combination 10 was chosen as the detection target. According to the analysis results, the model created had an average AUC of 0.92 (specificity: 96%; sensitivity: 81%) and had 80% sensitivity to stage-I malignant samples, 100% sensitivity to stage-II samples, and 100% sensitivity to stage-III samples.

Another modeling analysis was performed based on lung nodule tissue samples in which molecular marker combination 11 was chosen as the detection target. According to the analysis results, the model created had an average AUC of 0.88 (specificity: 81%; sensitivity: 86%) and had 86% sensitivity to stage-I malignant samples, 100% sensitivity to stage-II samples, and 100% sensitivity to stage-III samples.

Another modeling analysis was performed based on lung nodule tissue samples in which molecular marker combination 12 was chosen as the detection target. According to the analysis results, the model created had an average AUC of 0.81 (specificity: 82%; sensitivity: 75%) and had 74% sensitivity to stage-I malignant samples, 100% sensitivity to stage-II samples, and 100% sensitivity to stage-III samples.

Another modeling analysis was performed based on lung nodule tissue samples in which molecular marker combination 13 was chosen as the detection target. According to the analysis results, the model created had an average AUC of 0.79 (specificity: 82%; sensitivity: 75%) and had 75% sensitivity to stage-I malignant samples, 100% sensitivity to stage-II samples, and 100% sensitivity to stage-III samples.

Another modeling analysis was performed based on lung nodule tissue samples in which molecular marker combination 14 was chosen as the detection target. According to the analysis results, the model created had an average AUC of 0.82 (specificity: 79%; sensitivity: 81%) and had 80% sensitivity to stage-I malignant samples, 100% sensitivity to stage-II samples, and 100% sensitivity to stage-III samples.

Another modeling analysis was performed based on lung nodule tissue samples in which molecular marker combination 15 was chosen as the detection target. According to the analysis results, the model created had an average AUC of 0.91 (specificity: 96%; sensitivity: 79%) and had 79% sensitivity to stage-I malignant samples, 100% sensitivity to stage-II samples, and 100% sensitivity to stage-III samples.

Another modeling analysis was performed based on lung nodule tissue samples in which molecular marker combination 16 was chosen as the detection target. According to the analysis results, the model created had an average AUC of 0.81 (specificity: 75%; sensitivity: 81%) and had 80% sensitivity to stage-I malignant samples, 100% sensitivity to stage-II samples, and 100% sensitivity to stage-III samples.

Another modeling analysis was performed based on lung nodule tissue samples in which molecular marker combination 17 was chosen as the detection target. According to the analysis results, the model created had an average AUC of 0.92 (specificity: 96%; sensitivity: 81%) and had 80% sensitivity to stage-I malignant samples, 100% sensitivity to stage-II samples, and 100% sensitivity to stage-III samples.

The ROC curves of the aforesaid molecular marker combinations in this embodiment are shown in FIG. 1. It can be known from the foregoing experimental results that certain molecular markers had better performance when used together than when used individually. For example, in this embodiment, each of molecular marker combinations 1-2, 4-11, 14, 15, and 17 had a greater AUC, SP (specificity), or SN (sensitivity) value as a whole than its individual constituent Markers. That certain molecular markers chosen for the present invention by the inventor have higher sensitivity and specificity when used in appropriate combinations than when used alone has potential value in clinical applications.

### Embodiment 5: Performances in Detecting the Benignity or Malignancy of Lung Nodules By Six Molecular Markers and Combinations Thereof in Respiratory Tract Fluid Samples

In this embodiment, the foregoing six molecular markers and combinations thereof in 173 respiratory tract fluid samples were detected. 65 of the samples had been determined as benign by surgical biopsy, and the remaining 108 samples malignant. The malignant samples included 33 stage-I samples, four stage-II samples, four stage-III samples, 10 stage-IV samples, and 57 other malignant samples. The detection reagent kit, detection method, and data analysis criteria used in this embodiment were the same as those in embodiment 2. The primer and probe combinations employed were the preferred combinations in embodiment 1.

Single-molecular-marker detection was performed on Marker1, Marker2, Marker3, Marker4, Marker5, and Marker6 in the respiratory tract fluid samples. All the six molecular markers are highly correlated to lung cancer. The performance of each Marker's being individually detected in a respiratory tract fluid sample is summarized in Table 9 The AUC value corresponding to each individual Marker ranged from 0.66 to 0.79 (i.e., <0.8).

For more detailed detection results, please refer to Table 9.

TABLE 9

| | | 95% CI | | Youden Cutoff | |
|---|---|---|---|---|---|
| | AUC | AUC.lower | AUC.upper | Sensitivity | Specificity |
| Marker1 | 0.77 | 0.70 | 0.84 | 0.69 | 0.81 |
| Marker2 | 0.79 | 0.73 | 0.85 | 0.73 | 0.76 |
| Marker3 | 0.66 | 0.58 | 0.73 | 0.35 | 0.97 |
| Marker4 | 0.76 | 0.69 | 0.83 | 0.83 | 0.58 |
| Marker5 | 0.75 | 0.69 | 0.82 | 0.54 | 0.94 |
| Marker6 | 0.73 | 0.65 | 0.80 | 0.50 | 0.91 |

In this embodiment, detection of different combinations of the six molecular markers was also performed. The samples, detection method, and data analysis method employed were the same as in embodiment 2.

The combinations of the molecular markers detected are detailed in Table 8.

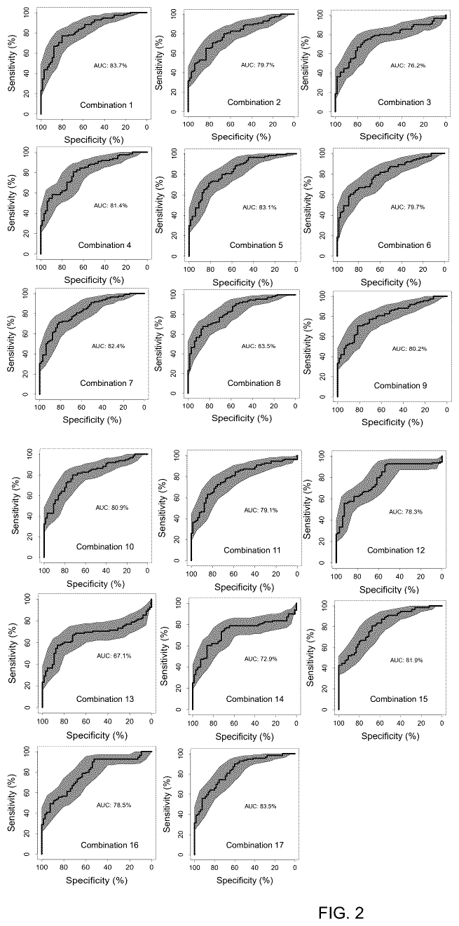

A modeling analysis was performed based on respiratory tract fluid samples in which molecular marker combination 1 was chosen as the detection target. According to the analysis results, the model created had an average AUC of 0.84 (specificity: 80%; sensitivity: 77%) and had 68% sensitivity to stage-I malignant samples, 75% sensitivity to stage-II samples, 100% sensitivity to stage-III samples, and 88% sensitivity to stage-IV samples. The corresponding ROC curve is shown in FIG. 2.

Another modeling analysis was performed based on respiratory tract fluid samples in which molecular marker combination 2 was chosen as the detection target. According to the analysis results, the model created had an average AUC of 0.80 (specificity: 83%; sensitivity: 60%) and had 42% sensitivity to stage-I malignant samples, 50% sensitivity to stage-II samples, 100% sensitivity to stage-III samples, and 81% sensitivity to stage-IV samples. The corresponding ROC curve is shown in FIG. 2.

Another modeling analysis was performed based on respiratory tract fluid samples in which molecular marker combination 3 was chosen as the detection target. According to the analysis results, the model created had an average AUC of 0.77 (specificity: 71%; sensitivity: 75%) and had 58% sensitivity to stage-I malignant samples, 100% sensitivity to stage-II samples, 88% sensitivity to stage-III samples, and 88% sensitivity to stage-IV samples. The corresponding ROC curve is shown in FIG. 2.

Another modeling analysis was performed based on respiratory tract fluid samples in which molecular marker combination 4 was chosen as the detection target. According to the analysis results, the model created had an average AUC of 0.81 (specificity: 69%; sensitivity: 81%) and had 74% sensitivity to stage-I malignant samples, 75% sensitivity to stage-II samples, 88% sensitivity to stage-III samples, and 88% sensitivity to stage-IV samples. The corresponding ROC curve is shown in FIG. 2.

Another modeling analysis was performed based on respiratory tract fluid samples in which molecular marker combination 5 was chosen as the detection target. According to the analysis results, the model created had an average AUC of 0.83 (specificity: 82%; sensitivity: 71%) and had 47% sensitivity to stage-I malignant samples, 100% sensitivity to stage-II samples, 100% sensitivity to stage-III samples, and 88% sensitivity to stage-IV samples. The corresponding ROC curve is shown in FIG. 2.

Another modeling analysis was performed based on respiratory tract fluid samples in which molecular marker combination 6 was chosen as the detection target. According to the analysis results, the model created had an average AUC of 0.80 (specificity: 88%; sensitivity: 61%) and had 45% sensitivity to stage-I malignant samples, 50% sensitivity to stage-II samples, 100% sensitivity to stage-III samples, and 79% sensitivity to stage-IV samples. The corresponding ROC curve is shown in FIG. 2.

Another modeling analysis was performed based on respiratory tract fluid samples in which molecular marker combination 7 was chosen as the detection target. According to the analysis results, the model created had an average AUC of 0.82 (specificity: 82%; sensitivity: 71%) and had 58% sensitivity to stage-I malignant samples, 75% sensitivity to stage-II samples, 88% sensitivity to stage-III samples, and 86% sensitivity to stage-IV samples. The corresponding ROC curve is shown in FIG. 2.

Another modeling analysis was performed based on respiratory tract fluid samples in which molecular marker combination 8 was chosen as the detection target. According to the analysis results, the model created had an average AUC of 0.84 (specificity: 86%; sensitivity: 68%) and had 53% sensitivity to stage-I malignant samples, 50% sensitivity to stage-II samples, 88% sensitivity to stage-III samples, and 83% sensitivity to stage-IV samples. The corresponding ROC curve is shown in FIG. 2.

Another modeling analysis was performed based on respiratory tract fluid samples in which molecular marker combination 9 was chosen as the detection target. According to the analysis results, the model created had an average AUC of 0.80 (specificity: 82%; sensitivity: 70%) and had 50% sensitivity to stage-I malignant samples, 75% sensitivity to stage-II samples, 100% sensitivity to stage-III samples, and 86% sensitivity to stage-IV samples. The corresponding ROC curve is shown in FIG. 2.

Another modeling analysis was performed based on respiratory tract fluid samples in which molecular marker combination 10 was chosen as the detection target. According to the analysis results, the model created had an average AUC of 0.81 (specificity: 71%; sensitivity: 80%) and had 66% sensitivity to stage-I malignant samples, 50% sensitivity to stage-II samples, 100% sensitivity to stage-III samples, and 93% sensitivity to stage-IV samples.

Another modeling analysis was performed based on respiratory tract fluid samples in which molecular marker combination 11 was chosen as the detection target. According to the analysis results, the model created had an average AUC of 0.79 (specificity: 55%; sensitivity: 85%) and had 71% sensitivity to stage-I malignant samples, 100% sensitivity to stage-II samples, 88% sensitivity to stage-III samples, and 95% sensitivity to stage-IV samples.

Another modeling analysis was performed based on respiratory tract fluid samples in which molecular marker combination 12 was chosen as the detection target. According to the analysis results, the model created had an average AUC of 0.78 (specificity: 54%; sensitivity: 91%) and had 87% sensitivity to stage-I malignant samples, 100% sensitivity to stage-II samples, 88% sensitivity to stage-III samples, and 95% sensitivity to stage-IV samples.

Another modeling analysis was performed based on respiratory tract fluid samples in which molecular marker combination 13 was chosen as the detection target. According to the analysis results, the model created had an average AUC of 0.67 (specificity: 86%; sensitivity: 57%) and had 63% sensitivity to stage-I malignant samples, 75% sensitivity to stage-II samples, 75% sensitivity to stage-III samples, and 79% sensitivity to stage-IV samples.

Another modeling analysis was performed based on respiratory tract fluid samples in which molecular marker combination 14 was chosen as the detection target. According to the analysis results, the model created had an average AUC of 0.73 (specificity: 86%; sensitivity: 60%) and had 39% sensitivity to stage-I malignant samples, 75% sensitivity to stage-II samples, 75% sensitivity to stage-III samples, and 81% sensitivity to stage-IV samples.

Another modeling analysis was performed based on respiratory tract fluid samples in which molecular marker combination 15 was chosen as the detection target. According to the analysis results, the model created had an average AUC of 0.82 (specificity: 68%; sensitivity: 81%) and had 74% sensitivity to stage-I malignant samples, 75% sensitivity to stage-II samples, 88% sensitivity to stage-III samples, and 88% sensitivity to stage-IV samples.

Another modeling analysis was performed based on respiratory tract fluid samples in which molecular marker combination 16 was chosen as the detection target. According to the analysis results, the model created had an average AUC of 0.79 (specificity: 52%; sensitivity: 93%) and had 87% sensitivity to stage-I malignant samples, 100% sensitivity to stage-II samples, 88% sensitivity to stage-III samples, and 93% sensitivity to stage-IV samples.

Another modeling analysis was performed based on respiratory tract fluid samples in which molecular marker combination 17 was chosen as the detection target. According to the analysis results, the model created had an average AUC of 0.84 (specificity: 60%; sensitivity: 90%) and had 79% sensitivity to stage-I malignant samples, 100% sensitivity to stage-II samples, 100% sensitivity to stage-III samples, and 95% sensitivity to stage-IV samples.

The ROC curves of the aforesaid molecular marker combinations in this embodiment are shown in FIG. 2. It can be known from the foregoing experimental results that certain molecular markers had better performance when used together than when used individually. For example, in this embodiment, each of molecular marker combinations 1-11 and 15-17 had better performance (e.g., in AUC, SP, or SN) as a whole than its individual constituent Markers. That certain molecular markers chosen for the present invention by the inventor have higher sensitivity and specificity when used in appropriate combinations than when used alone has potential value in clinical applications.

### Embodiment 6: Performances in Detecting the Benignity or Malignancy of Lung Nodules by Six Molecular Markers and Combinations Thereof in Respiratory Tract Fluid Samples in Independent Validation Sets

In this embodiment, the foregoing six molecular markers and combinations thereof in 61 naturally enrolled respiratory tract fluid samples were detected. 19 of the samples had been determined as benign by surgical biopsy, and the remaining 42 samples malignant (the stage of each malignant sample being unknown). The detection reagent kit, detection method, and data analysis criteria used in this embodiment were the same as those in embodiment 2. The primer and probe combinations employed were the preferred combinations in embodiment 1.

Single-molecular-marker detection was performed on Marker1, Marker2, Marker3, Marker4, Marker5, and Marker6 in the respiratory tract fluid samples. The performance of each Marker's being individually detected in a respiratory tract fluid sample is summarized in Table 10. The AUC value corresponding to each individual Marker ranged from 0.75 to 0.86 (i.e., <0.87).

For more detailed detection results, please refer to Table 10.

TABLE 10

| | | 95% CI | | Youden Cutoff | |
|---|---|---|---|---|---|
| | AUC | AUC.lower | AUC.upper | Sensitivity | Specificity |
| Marker1 | 0.7973 | 0.70 | 0.90 | 61.36 | 94.44 |
| Marker2 | 0.8636 | 0.78 | 0.95 | 63.64 | 94.44 |
| Marker3 | 0.8548 | 0.76 | 0.95 | 61.36 | 94.44 |
| Marker4 | 0.8636 | 0.77 | 0.95 | 59.09 | 94.44 |
| Marker5 | 0.859 | 0.75 | 0.93 | 75 | 83.70 |
| Marker6 | 0.7519 | 0.63 | 0.87 | 47.73 | 94.44 |

In this embodiment, detection of different combinations of the six molecular markers was also performed. The samples, detection method, and data analysis method employed were the same as in embodiment 2.

The combinations of the molecular markers detected are detailed in Table 8.

A modeling analysis was performed based on respiratory tract fluid samples in which molecular marker combination 1 was chosen as the detection target. According to the analysis results, the model created had an average AUC of 0.919 (specificity: 94.4%; sensitivity: 79.5%).

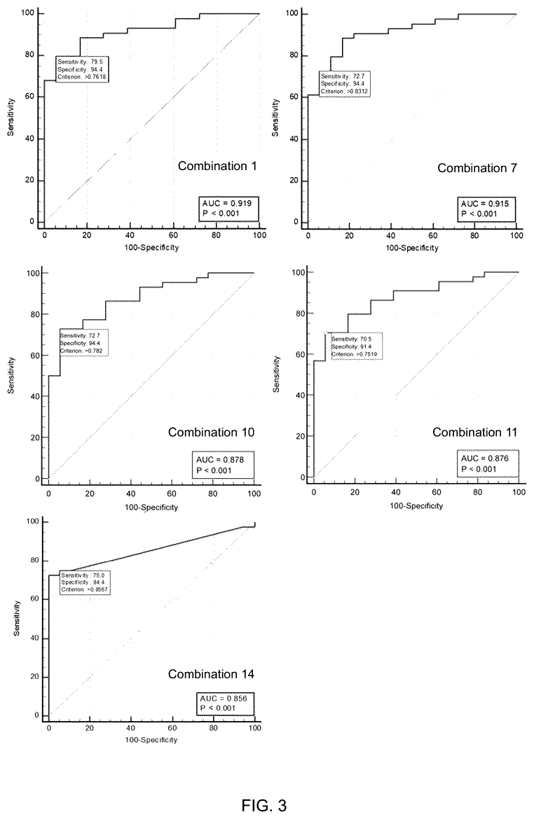

The corresponding ROC curve is shown in FIG. 3.

Another modeling analysis was performed based on respiratory tract fluid samples in which molecular marker combination 7 was chosen as the detection target. According to the analysis results, the model created had an average AUC of 0.915 (specificity: 94.4%; sensitivity: 72.7%). The corresponding ROC curve is shown in FIG. 3.

Another modeling analysis was performed based on respiratory tract fluid samples in which molecular marker combination 10 was chosen as the detection target. According to the analysis results, the model created had an average AUC of 0.878 (specificity: 94.4%; sensitivity: 72.7%). The corresponding ROC curve is shown in FIG. 3.

Another modeling analysis was performed based on respiratory tract fluid samples in which molecular marker combination 11 was chosen as the detection target. According to the analysis results, the model created had an average AUC of 0.876 (specificity: 91.4%; sensitivity: 70.5%). The corresponding ROC curve is shown in FIG. 3.

It can be known from the foregoing experimental results that certain molecular markers had better performance when used together than when used individually. For example, in this embodiment, each of molecular marker combinations 1, 7, 10, 11, and 14 (see FIG. 3) had better performance (e.g., in AUC, SP, or SN) as a whole than its individual constituent Markers. All the Marker combinations led to greater AUC values than individual Markers. Each of the high-specificity (SP>90) combinations 1, 7, 10, and 14 had higher sensitivity as a whole than its individual constituent Markers, and combination 1 gave better overall performance than combination 7 by having 6% higher sensitivity than combination 7 while maintaining its high specificity (SP>90). Each of combinations 10, 11, and 14 included only two Markers but still had better performance (demonstrated by a greater AUC value and higher sensitivity) than its individual constituent Markers. Moreover, given the same sensitivity level (with SN being about 75), the two-Marker models had higher specificity than the model using only Marker5. That certain molecular markers chosen for the present invention by the inventor have higher sensitivity and specificity when used in appropriate combinations than when used alone has potential value in clinical applications.

The embodiments described above disclose only a few implementation modes of the present invention. The relatively specific and detailed description of those embodiments shall not be understood as restrictive of the scope of the invention. It should be pointed out that a person of ordinary skill in the art may make various changes and improvements without departing from the concept of the invention, and that all such changes and improvements shall fall within the scope of patent protection for the invention. The scope of patent protection for the invention is defined by the appended claims.

1. A molecular marker for identifying DNA methylation and thereby detecting benignity or malignancy of a lung nodule, characterized in that the molecular marker for identifying DNA methylation comprises the sequence of, or the completely complementary sequence to, SEQ ID NO:6 or comprises a continuous fragment of at least 55% of the full length of the sequence of, or the completely complementary sequence to, SEQ ID NO:6.

2. The molecular marker for identifying DNA methylation and thereby detecting benignity or malignancy of a lung nodule as claimed in claim 1, characterized by further comprising at least one selected from the group consisting of the sequences of, or the completely complementary sequences to, SEQ ID NO:1 to SEQ ID NO:5 or at least one selected from the group consisting of continuous fragments each of at least 55% of the full length of one of the sequences of, or the completely complementary sequences to, SEQ ID NO:1 to SEQ ID NO:5.

3. The molecular marker for identifying DNA methylation and thereby detecting benignity or malignancy of a lung nodule as claimed in claim 2, characterized in that the molecular marker for identifying DNA methylation comprises the sequences of, or the completely complementary sequences to, SEQ ID NO:6 and SEQ ID NO:4; or comprises continuous fragments each of at least 55% of the full length of one of the sequences of, or the completely complementary sequences to, SEQ ID NO:6 and SEQ ID NO:4.

4. The molecular marker for identifying DNA methylation and thereby detecting benignity or malignancy of a lung nodule as claimed in claim 3, characterized in that the molecular marker for identifying DNA methylation further comprises the sequence of, or the completely complementary sequence to, SEQ ID NO:2; or further comprises a continuous fragment of at least 55% of the full length of the sequence of, or the completely complementary sequence to, SEQ ID NO:2.

5. The molecular marker for identifying DNA methylation and thereby detecting benignity or malignancy of a lung nodule as claimed in claim 1 or 3, characterized in that the molecular marker for identifying DNA methylation further comprises the sequence of, or the completely complementary sequence to, SEQ ID NO:5; or
further comprises a continuous fragment of at least 55% of the full length of the sequence of, or the completely complementary sequence to, SEQ ID NO:5.

6. The molecular marker for identifying DNA methylation and thereby detecting benignity or malignancy of a lung nodule as claimed in claim 5, characterized by further comprising the sequences of, or the completely complementary sequences to, SEQ ID NO:2 and/or SEQ ID NO:3; or
further comprising continuous fragments each of at least 55% of the full length of one of the sequences of, or the completely complementary sequences to, SEQ ID NO:2 and/or SEQ ID NO:3.

7. The molecular marker for identifying DNA methylation and thereby detecting benignity or malignancy of a lung nodule as claimed in claim 5, characterized by further comprising the sequences of, or the completely complementary sequences to, SEQ ID NO:1 and/or SEQ ID NO:3; or
further comprising continuous fragments each of at least 55% of the full length of one of the sequences of, or the completely complementary sequences to, SEQ ID NO:1 and/or SEQ ID NO:3.

8. The molecular marker for identifying DNA methylation and thereby detecting benignity or malignancy of a lung nodule as claimed in claim 2, characterized in that the molecular marker for identifying DNA methylation comprises the sequences of, or the completely complementary sequences to, SEQ ID NO:6 and SEQ ID NO:1; or
the molecular marker for identifying DNA methylation comprises the sequences of, or the completely complementary sequences to, SEQ ID NO:6 and SEQ ID NO:2; or
the molecular marker for identifying DNA methylation comprises the sequences of, or the completely complementary sequences to, SEQ ID NO:6, SEQ ID NO:1, and SEQ ID NO:2; or
the molecular marker for identifying DNA methylation comprises the sequences of, or the completely complementary sequences to, SEQ ID NO:6 and SEQ ID NO:3; or
the molecular marker for identifying DNA methylation comprises the sequences of, or the completely complementary sequences to, SEQ ID NO:6 and SEQ ID NO:5; or
the molecular marker for identifying DNA methylation comprises continuous fragments each of at least 55% of the full length of one of the sequences of, or the completely complementary sequences to, SEQ ID NO:6 and SEQ ID NO:1; or SEQ ID NO:6 and SEQ ID NO:2; or SEQ ID NO:6, SEQ ID NO:1, and SEQ ID NO:2; or SEQ ID NO:6 and SEQ ID NO:3; or SEQ ID NO:6 and SEQ ID NO:5.

9. The molecular marker for identifying DNA methylation and thereby detecting benignity or malignancy of a lung nodule as claimed in claim 2, characterized in that the molecular marker for identifying DNA methylation comprises the sequences of, or the completely complementary sequences to, SEQ ID NO:6, SEQ ID NO:1, SEQ ID NO:2, SEQ ID NO:3, and SEQ ID NO:4; or
comprises continuous fragments each of at least 55% of the full length of one of the sequences of, or the completely complementary sequences to, SEQ ID NO:6, SEQ ID NO:1, SEQ ID NO:2, SEQ ID NO:3, and SEQ ID NO:4.

10. The molecular marker for identifying DNA methylation and thereby detecting benignity or malignancy of a lung nodule as claimed in claim 2, characterized in that the molecular marker for identifying DNA methylation comprises the sequences of, or the completely complementary sequences to, SEQ ID NO:6, SEQ ID NO:1, SEQ ID NO:3, SEQ ID NO:4, and SEQ ID NO:5; or
comprises continuous fragments each of at least 55% of the full length of one of the sequences of, or the completely complementary sequences to, SEQ ID NO:6, SEQ ID NO:1, SEQ ID NO:3, SEQ ID NO:4, and SEQ ID NO:5.

11. The molecular marker for identifying DNA methylation and thereby detecting benignity or malignancy of a lung nodule as claimed in claim 2, characterized in that the molecular marker for identifying DNA methylation comprises the sequences of, or the completely complementary sequences to, SEQ ID NO:1 to SEQ ID NO:6; or
comprises continuous fragments each of at least 55% of the full length of one of the sequences of, or the completely complementary sequences to, SEQ ID NO:1 to SEQ ID NO:6.

12. A molecular marker for identifying DNA methylation and thereby detecting benignity or malignancy of a lung nodule, characterized in that the molecular marker for identifying DNA methylation comprises the sequences of, or the completely complementary sequences to, SEQ ID NO:2 and SEQ ID NO:4; or
the molecular marker for identifying DNA methylation comprises the sequences of, or the completely complementary sequences to, SEQ ID NO:1 to SEQ ID NO:4; or
the molecular marker for identifying DNA methylation comprises the sequences of, or the completely complementary sequences to, SEQ ID NO:1 to SEQ ID NO:5; or
the molecular marker for identifying DNA methylation comprises the sequences of, or the completely complementary sequences to, SEQ ID NO:3 and SEQ ID NO:6; or
the molecular marker for identifying DNA methylation comprises continuous fragments each of at least 55% of the full length of one of the sequences of, or the completely complementary sequences to, SEQ ID NO:2 and SEQ ID NO:4; or SEQ ID NO:1 to SEQ ID NO:4; or SEQ ID NO:1 to SEQ ID NO:5; or SEQ ID NO:3 and SEQ ID NO:6.

13. The molecular marker for identifying DNA methylation and thereby detecting benignity or malignancy of a lung nodule as claimed in any of claims 1 to 12, characterized in that:
the molecular marker for identifying DNA methylation is the following continuous fragments each of at least 55% of the full length of one of the sequences of SEQ ID NO:1 to SEQ ID NO:6:
the sequence corresponding to a fragment amplified from SEQ ID NO:1 using SEQ ID NO:7 and SEQ ID NO:8, or SEQ ID NO:10 and SEQ ID NO:11, or SEQ ID NO:13 and SEQ ID NO:14 as primers; and/or
the sequence corresponding to a fragment amplified from SEQ ID NO:2 using SEQ ID NO:16 and SEQ ID NO:17, or SEQ ID NO:19 and SEQ ID NO:20, or SEQ ID NO:22 and SEQ ID NO:23 as primers; and/or
the sequence corresponding to a fragment amplified from SEQ ID NO:3 using SEQ ID NO:25 and SEQ ID NO:26, or SEQ ID NO:28 and SEQ ID NO:29, or SEQ ID NO:31 and SEQ ID NO:32 as primers; and/or
the sequence corresponding to a fragment amplified from SEQ ID NO:4 using SEQ ID NO:34 and SEQ ID NO:35, or SEQ ID NO:37 and SEQ ID NO:38, or SEQ ID NO:40 and SEQ ID NO:41 as primers; and/or
the sequence corresponding to a fragment amplified from SEQ ID NO:5 using SEQ ID NO:43 and SEQ ID NO:44, or SEQ ID NO:46 and SEQ ID NO:47, or SEQ ID NO:49 and SEQ ID NO:50 as primers; and/or
the sequence corresponding to a fragment amplified from SEQ ID NO:6 using SEQ ID NO:52 and SEQ ID NO:53, or SEQ ID NO:55 and SEQ ID NO:56, or SEQ ID NO:58 and SEQ ID NO:59 as primers.

14. The molecular marker for identifying DNA methylation and thereby detecting benignity or malignancy of a lung nodule as claimed in claim 13, characterized in that:
the molecular marker for identifying DNA methylation is the following continuous fragments each of at least 55% of the full length of one of the sequences of SEQ ID NO:1 to SEQ ID NO:6:
the sequence corresponding to the fragment amplified from SEQ ID NO:1 using SEQ ID NO:7 and SEQ ID NO:8 as primers; and/or
the sequence corresponding to the fragment amplified from SEQ ID NO:2 using SEQ ID NO:16 and SEQ ID NO:17 as primers; and/or
the sequence corresponding to the fragment amplified from SEQ ID NO:3 using SEQ ID NO:28 and SEQ ID NO:29 as primers; and/or
the sequence corresponding to the fragment amplified from SEQ ID NO:4 using SEQ ID NO:37 and SEQ ID NO:38 as primers; and/or
the sequence corresponding to the fragment amplified from SEQ ID NO:5 using SEQ ID NO:43 and SEQ ID NO:44 as primers; and/or
the sequence corresponding to the fragment amplified from SEQ ID NO:6 using SEQ ID NO:52 and SEQ ID NO:53 as primers.

15. The molecular marker for identifying DNA methylation and thereby detecting benignity or malignancy of a lung nodule as claimed in any of claims 1 to 14, characterized in that the molecular marker for identifying DNA methylation is a molecular marker to be detected in a respiratory tract sample selected from the group consisting of a lung tissue sample and a respiratory tract fluid sample.

16. A use of the molecular marker for identifying DNA methylation as claimed in any of claims 1 to 15 and/or a reagent for detecting a methylation level thereof in preparing a reagent kit for detecting benignity or malignancy of a lung nodule and/or lung cancer.

17. A reagent kit for detecting benignity or malignancy of a lung nodule, characterized in that the reagent kit comprises a reagent for detecting a methylation level of the molecular marker for identifying DNA methylation as claimed in any of claims 1 to 13.

18. The reagent kit for detecting benignity or malignancy of a lung nodule as claimed in claim 17, characterized in that the reagent of the reagent kit is for use in a polymerase chain reaction (PCR) amplification method, a quantitative fluorescent PCR method, a digital PCR method, a liquid chip method, a first-generation sequencing method, a third-generation sequencing method, a second-generation sequencing method, a pyrosequencing method, a bisulfite conversion-based sequencing method, a methylation chip method, a reduced-representation bisulfite sequencing method, or a combination of the aforesaid methods.

19. The reagent kit for detecting benignity or malignancy of a lung nodule as claimed in claim 18, characterized in that the reagent comprises the following combinations of primers and probe for performing quantitative fluorescent PCR detection on the molecular marker for identifying DNA methylation:
for SEQ ID NO:1, at least one combination selected from the group consisting of: primers with the sequences of SEQ ID NO:7 and SEQ ID NO:8 respectively and a probe with the sequence of SEQ ID NO:9; primers with the sequences of SEQ ID NO:10 and SEQ ID NO:11 respectively and a probe with the sequence of SEQ ID NO:12; and primers with the sequences of SEQ ID NO:13 and SEQ ID NO:14 respectively and a probe with the sequence of SEQ ID NO:15; and/or
for SEQ ID NO:2, at least one combination selected from the group consisting of: primers with the sequences of SEQ ID NO:16 and SEQ ID NO:17 respectively and a probe with the sequence of SEQ ID NO:18; primers with the sequences of SEQ ID NO:19 and SEQ ID NO:20 respectively and a probe with the sequence of SEQ ID NO:21; and primers with the sequences of SEQ ID NO: 22 and SEQ ID NO:23 respectively and a probe with the sequence of SEQ ID NO:24; and/or
for SEQ ID NO:3, at least one combination selected from the group consisting of: primers with the sequences of SEQ ID NO:25 and SEQ ID NO:26 respectively and a probe with the sequence of SEQ ID NO:27; primers with the sequences of SEQ ID NO:28 and SEQ ID NO:29 respectively and a probe with the sequence of SEQ ID NO:30; and primers with the sequences of SEQ ID NO:31 and SEQ ID NO:32 respectively and a probe with the sequence of SEQ ID NO:33; and/or
for SEQ ID NO:4, at least one combination selected from the group consisting of: primers with the sequences of SEQ ID NO:34 and SEQ ID NO:35 respectively and a probe with the sequence of SEQ ID NO:36; primers with the sequences of SEQ ID NO:37 and SEQ ID NO:38 respectively and a probe with the sequence of SEQ ID NO:39; and primers with the sequences of SEQ ID NO:40 and SEQ ID NO:41 respectively and a probe with the sequence of SEQ ID NO:42; and/or
for SEQ ID NO:5, at least one combination selected from the group consisting of: primers with the sequences of SEQ ID NO:43 and SEQ ID NO:44 respectively and a probe with the sequence of SEQ ID NO:45; primers with the sequences of SEQ ID NO:46 and SEQ ID NO:47 respectively and a probe with the sequence of SEQ ID NO:48; and primers with the sequences of SEQ ID NO:49 and SEQ ID NO:50 respectively and a probe with the sequence of SEQ ID NO:51; and/or
for SEQ ID NO:6, at least one combination selected from the group consisting of: primers with the sequences of SEQ ID NO:52 and SEQ ID NO:53 respectively and a probe with the sequence of SEQ ID NO:54; primers with the sequences of SEQ ID NO:55 and SEQ ID NO:56 respectively and a probe with the sequence of SEQ ID NO:57; and primers with the sequences of SEQ ID NO:58 and SEQ ID NO:59 respectively and a probe with the sequence of SEQ ID NO:60; or
at least one combination of primers and probe each having a plurality of continuous nucleotides with at least 70%, 80%, 90%, 95%, or 99% sequence identity to the sequence of one or the other primer or the probe in any of the foregoing combinations.

20. The reagent kit for detecting benignity or malignancy of a lung nodule as claimed in claim 19, characterized in that the reagent comprises the following combinations of primers and probe:
for SEQ ID NO:1, the primers with the sequences of SEQ ID NO:7 and SEQ ID NO:8 respectively and the probe with the sequence of SEQ ID NO:9; and/or
for SEQ ID NO:2, the primers with the sequences of SEQ ID NO:16 and SEQ ID NO:17 respectively and the probe with the sequence of SEQ ID NO:18; and/or
for SEQ ID NO:3, the primers with the sequences of SEQ ID NO:28 and SEQ ID NO:29 respectively and the probe with the sequence of SEQ ID NO:30; and/or
for SEQ ID NO:4, the primers with the sequences of SEQ ID NO:37 and SEQ ID NO:38 respectively and the probe with the sequence of SEQ ID NO:39; and/or
for SEQ ID NO:5, the primers with the sequences of SEQ ID NO:43 and SEQ ID NO:44 respectively and the probe with the sequence of SEQ ID NO:45; and/or
for SEQ ID NO:6, the primers with the sequences of SEQ ID NO:52 and SEQ ID NO:53 respectively and the probe with the sequence of SEQ ID NO:54.

21. The reagent kit for detecting benignity or malignancy of a lung nodule as claimed in any of claims 17 to 20, characterized in that the reagent kit further comprises primers and a probe for performing quantitative fluorescent PCR detection on a reference gene ACTB.

22. The reagent kit for detecting benignity or malignancy of a lung nodule as claimed in claim 21, characterized in that the primers and the probe for the reference gene ACTB are: primers with the sequences of SEQ ID NO:61 and SEQ ID NO:62 respectively and a probe with the sequence of SEQ ID NO:63.

23. The reagent kit for detecting benignity or malignancy of a lung nodule as claimed in any of claims 17 to 20, characterized in that the reagent kit is applied to a test sample, the test sample being a respiratory tract sample selected from the group consisting of a lung tissue sample and a respiratory tract fluid sample.

24. A method for detecting benignity or malignancy of a lung nodule and/or lung cancer, characterized by comprising the steps of:
(1) extracting genomic DNA from a test sample;
(2) performing a bisulfite treatment on the extracted genomic DNA to obtain a converted DNA; and
(3) performing detection with the reagent kit of any of claims 17 to 21.
